(19) United States
(12) Patent Application Publication
(10) Pub. No.: US 2004/0110261 A1
(43) Pub. Date: Jun. 10, 2004

(54) Helicobacter dd-heptosyltransferase

(76) Inventors: Koji Hiratsuka, Stittsville (CA); Eleonora Altman, Gloucester (CA)

Correspondence Address: George A Seaby, Seaby & Associates, 603-880 Willington Street, Ottawa, ON, K1R 6K7, CA

(21) Appl. No.: 10/451,685

(22) PCT Filed: Jun. 28, 2001
(86) PCT No.: PCT/CA01/00969

(30) Foreign Application Priority Data
Jun. 28, 2000 (CA) CA00/00777

(51) Int. Cl. C07H 21/04; C12N 9/10; C12N 1/21

(52) U.S. Cl. 435/193; 435/69.1; 435/252.3; 435/320.1; 530/388.26; 536/23.2

(57) ABSTRACT

This invention relates to newly identified polynucleotides and polypeptides, and their production and uses, as well as their variants, agonists and antagonists, and their uses. In particular, the invention relates to novel heptosyltransferase polynucleotides and polypeptides.

### FIELD OF THE INVENTION

This invention relates to newly identified polynucleotides and polypeptides, and their production and uses, as well as their variants, agonists and antagonists, and their uses. In particular, the invention relates to novel heptosyltransferase polynucleotides and polypeptides.

### BACKGROUND OF THE INVENTION

Helicobacter pylori is a spiral Gram negative bacterium which colonizes the human stomach. It is estimated that up to 50% of the human population is infected with H. pylori (Dunn et al., 1997). Thus, H. pylori remains one of the most prevalent bacterial pathogens worldwide.

Infection by H. pylori is associated with chronic superficial and active gastritis (Blaser, 1990), which may eventually develop into peptic ulcers (Blaser, 1995; Graham, 1991). Furthermore, prolonged infection by H. pylori can lead to the development of gastric carcinoma and mucosa-associated lymphoid tissue (MALT) lymphoma (Dunn, et al, 1997; Parsonnet, et al, 1994). H. pylori has been declared a human carcinogen by the International Agency for Cancer Research. Many research initiatives worldwide are aimed at determining the reasons why H. pylori produces such a variety of pathogenic outcomes.

Genetic variations in both the host and pathogen likely explain much of the clinical variation. Genomic mapping of several H. pylori strains showed a variation in the arrangement of several genetic markers (Jiang et al, 1996). This variability was also observed in gene organization, gene content and nucleotide sequence between the genome sequences of two H. pylori strains that have been determined and annotated (Tombs, et al., 1997; Alm, et al., 1999). Several factors associated with pathogenicity have been identified, including urease, vacuolating cytotoxin (VAC), cytotoxin associated gene (CAG), various adhesins, iron-binding proteins, catalase, superoxide dismutase and lipopolysaccharide (LPS)(for review see Dunn et al., 1997).

The LPS of H. pylori may play several roles in pathogenesis. In particular, H. pylori LPS has been implicated in causing abnormal acid secretion and in inducing apoptosis of epithelial cells and gastritis in mice (Piotrowski, et al., 1997a; Sakagami, et al., 1997; Kidd, et al., 1997; Piotrowski, et al., 1997b; Ootsubo, et al., 1997; Okumura, et al., 1998). H. pylori LPS may also be involved in triggering inflammatory response. Additionally, some strains of H. pylori express O-antigen polysaccharide chains which mimic Lewis blood group antigens (Aspinall, et al., 1997; Monteiro et al., 1998b) which are naturally expressed in the human gastric mucosa. Such antigenic mimicry may play a role in evasion of the host immune system. Alternatively, this mimicry may give rise to pathogenic autoimmune antibodies by the host (Appelmelk, et al., 1997). The exposure of LPS at the bacterial cell surface would make it an obvious putative colonization factor. Recently Edwards et al. (2000) showed that the O-chain polysaccharide found in the LPS of many strains of H. pylori may be involved in the adhesion of the pathogen to gastric epithelial cells. Similarly, Logan et al. (2000) have shown O-antigen to be an important H. pylori factor for the colonization of the murine stomach.

### SUMMARY OF THE INVENTION

Applicants have identified, cloned and characterized a gene involved in assembly of the core polysaccharide of the LPS molecule. The gene encodes a DD-heptosyltransferase (DDHepT) obtainable from Helicobacter that is responsible for adding DDHepII to the core LPS structure. When mutations were introduced into the gene, a truncated LPS with no O-antigen resulted. In addition, when mutations were introduced into the mouse-colonizing strain, H. pylori SS1, the mutant strain was unable to colonize the murine stomach.

A polypeptide of the invention is referred to herein as “DDHepT” or “DDHepT polypeptide” and a polynucleotide encoding a polypeptide of the invention is referred to herein as “DdhepT gene” or “DdhepT”

Broadly stated the present invention contemplates an isolated polynucleotide encoding a DD-HepT polypeptide of the invention, including mRNAs, DNAs, cDNAs, genomic DNAs, PNAs, as well as antisense analogs and biologically, diagnostically, prophylactically, clinically or therapeutically useful variants or fragments thereof, and compositions comprising same.

In particular, the present invention contemplates an isolated polynucleotide comprising a sequence that comprises at least 18 nucleotides and hybridizes under stringent conditions to the complementary nucleic acid sequence of SEQ. ID. NO. 1, 3, or 5 or a degenerate form thereof. In an embodiment the polynucleotide comprises a region encoding DD-HepT polypeptides comprising a sequence set out in SEQ ID NO: 1, 3, or 5 which includes a full length polynucleotide or a variant thereof. In a preferred embodiment the polynucleotide encodes a polynucleotide designated herein as HP0479.

The polynucleotides of the invention permit identification of untranslated nucleic acid sequences or regulatory sequences which specifically promote expression of genes operatively linked to the promoter regions. The invention therefore contemplates a polynucleotide encoding a regulatory sequence of a polynucleotide of the invention such as a promoter sequence, preferably a regulatory sequence of a DDhepT gene.

The polynucleotides encoding a mature polypeptide of the invention may include only the coding sequence for the mature polypeptide; the coding sequence for the mature polypeptide and additional coding sequences (e.g. leader or secretory sequences, proprotein sequences); the coding sequence for the mature polypeptide (and optionally additional coding sequences) and non-coding sequence, such as introns or non-coding sequences 5′ and/or 3′ of the coding sequence of the mature polypeptide.

The polynucleotides of the invention may be inserted into an appropriate expression vector, and the vector may contain the necessary elements for the transcription and translation of an inserted coding sequence. Accordingly, recombinant expression vectors may be constructed which comprise a polynucleotide of the invention, and where appropriate one or more transcription and translation elements linked to the polynucleotide.

Vectors are contemplated within the scope of the invention which comprise regulatory sequences of the invention, as well as chimeric gene constructs wherein a regulatory sequence of the invention is operably linked to a polynucleotide sequence encoding a heterologous protein (i.e. a protein not naturally expressed in the host cell), and a transcription termination signal.

A vector can be used to transform host cells to express a polypeptide of the invention, or a heterologous protein. Therefore, the invention further provides host cells containing a vector of the invention.

The invention also contemplates an isolated DD-HepT polypeptide encoded by a polynucleotide of the invention. In an embodiment, the invention provides a DD-HepT from Helicobacter comprising the amino acid sequence of SEQ ID NO:2, 4, or 6 or a variant thereof. Further embodiments of the invention provide biologically, diagnostically, prophylactically, clinically or therapeutically useful variants thereof and compositions comprising a polypeptide of the invention.

Among the embodiments of the invention are variants of a polypeptide of the invention encoded by naturally occurring alleles of a DDhepT gene.

Polypeptides of the invention may be obtained as an isolate from natural cell sources, but they are preferably produced by recombinant procedures. In one aspect the invention provides a method for preparing a polypeptide of the invention utilizing an isolated polynucleotide of the invention. In an embodiment a method for preparing a DDHepT polypeptide is provided comprising:

(a) transferring a recombinant expression vector of the invention having a polynucleotide sequence encoding a DD-HepT, into a host cell;

(b) selecting transformed host cells from untransformed host cells;

(c) culturing a selected transformed host cell under conditions which allow expression of the DD-HepT; and

(d) isolating the DD-HepT.

The invention further broadly contemplates a recombinant DD-HepT obtained using a method of the invention.

A polypeptide of the invention may be conjugated with other molecules, such as proteins, to prepare fusion proteins or chimeric proteins. This may be accomplished, for example, by the synthesis of N-terminal or C-terminal fusion proteins.

The invention further contemplates antibodies having specificity against an epitope of a polypeptide of the invention. Antibodies may be labeled with a detectable substance and used to detect polypeptides of the invention in biological samples, tissues, and cells.

The invention also permits the construction of nucleotide probes which are unique to the polynucleotides of the invention or to polypeptides of the invention. Therefore, the invention also relates to a probe comprising a sequence encoding a polypeptide of the invention, or a part thereof. The probe may be labeled, for example, with a detectable substance and it may be used to select from a mixture of nucleotide sequences a polynucleotide of the invention including polynucleotides encoding a polypeptide which displays one or more of the properties of a polypeptide of the invention.

In accordance with an aspect of the invention there is provided a method of, and products for, diagnosing and monitoring diseases by determining the presence of polynucleotides and polypeptides of the invention.

Still further the invention provides a method for evaluating a test compound or agent for its ability to modulate the activity of a polypeptide or polynucleotide of the invention. For example a substance which inhibits or enhances the catalytic activity of a polypeptide of the invention may be evaluated. “Modulate” refers to a change or an alteration in the biological activity of a polypeptide of the invention. Modulation may be an increase or a decrease in activity, a change in characteristics, or any other change in the biological, functional, or immunological properties of the polypeptide.

In an embodiment, the invention provides methods for identifying compounds which bind to or otherwise interact with and inhibit or activate an activity of a polypeptide or polynucleotide of the invention comprising:

(a) contacting a polypeptide or polynucleotide of the invention with a test compound under conditions to permit binding to or other interaction between the test compound and the polypeptide or polynucleotide to assess the binding to or other interaction with the test compound, wherein the binding or interaction is associated with a second component capable of providing a detectable signal in response to the binding or interaction of the polypeptide or polynucleotide with the test compound; and

(b) determining whether the test compound binds to or interacts with and activates or inhibits an activity of the polypeptide or polynucleotide by detecting the presence or absence of a signal generated from the binding or interaction of the test compound with the polypeptide or polynucleotide.

Compounds which modulate the biological activity of a polypeptide of the invention may also be identified using the methods of the invention by comparing the pattern and level of expression of a polynucleotide or polypeptide of the invention in cells and organisms, in the presence, and in the absence of the compounds.

Methods are also contemplated that identify compounds or substances (e.g. polypeptides) which bind to regulatory sequences (e.g. promoter sequences, enhancer sequences, negative modulator sequences).

Still another aspect of the invention provides a method of conducting a drug discovery business comprising:

(a) providing one or more systems or methods for identifying modulators of a polypeptide or polynucleotide of the invention;

(b) conducting therapeutic profiling of modulators identified in step (a), or further analogs thereof, for efficacy and toxicity in animals; and

(c) formulating a pharmaceutical composition including one or more modulators identified in step (b) as having an acceptable therapeutic profile.

In certain embodiments, the subject method may also include a step of establishing a distribution system for distributing the pharmaceutical composition for sale, and may optionally include establishing a sales group for marketing the pharmaceutical composition.

In yet another aspect of the invention, a method of conducting a target discovery business is provided comprising:

(a) providing one or more systems or methods for identifying modulators of a polypeptide or polynucleotide of the invention;

(b) optionally conducting therapeutic profiling of modulators identified in (a) for efficacy and toxicity in animals; and

(c) licensing to a third party the rights for further drug development and/or sales for modulators identified in step (a), or analogs thereof.

The substances and compounds identified using the methods of the invention, antibodies, and antisense polynucleotides may be used to modulate the biological activity of a polypeptide or polynucleotide of the invention, and they may be used in the prevention and treatment of disease. In an aspect of the invention the substances and compounds are inhibitors of polypeptides of the invention that are useful as antibacterial agents.

In accordance with an aspect of the invention there are provided agonists and antagonists of a DD-HepT, preferably bacteriostatic or bacteriocidal agonists or antagonists.

Accordingly, the polynucleotides and polypeptides of the invention, antibodies and substances and compounds may be formulated into compositions for administration to a cell or to a multicellular organism. Therefore, the present invention also relates to a composition comprising one or more of a polynucleotide or polypeptide of the invention, antibody or a substance or compound identified using the methods of the invention, and a pharmaceutically acceptable carrier, excipient or diluent. A method for treating or preventing a disease is also provided comprising administering to a patient in need thereof, a composition of the invention.

In accordance with certain embodiments of the invention, there are provided products, compositions and methods for assessing DDHepT expression, treating disease, assaying genetic variation, and administering a polypeptide or polynucleotide of the invention to an organism to raise an immunological response against a bacteria.

Having provided novel DDHepT polypeptides, and polynucleotides encoding same, the invention accordingly further provides methods for preparing oligosaccharides e.g. two or more saccharides. In specific embodiments, the invention relates to a method for preparing an oligosaccharide comprising contacting a reaction mixture comprising an activated D-glycero-α-D-manno-heptose (“DDHepII’), and an acceptor in the presence of a polypeptide of the invention.

In accordance with a further aspect of the invention, there are provided processes for utilizing polypeptides or polynucleotides of the invention, for in vitro purposes related to scientific research, synthesis of DNA, and manufacture of vectors.

In another embodiment of the invention there is provided a computer readable medium having stored thereon a member selected from the group consisting of: (a) a polynucleotide comprising the sequence of SEQ ID NO. 1, 3, or 5; (b) a polypeptide comprising the sequence of SEQ ID NO. 2, 4, or 6; (c) a data set of polynucleotide sequences wherein at least one of said sequences comprises the sequence of SEQ ID NO. 1, 3, or 5; (d) a data set of polypeptide sequences wherein at least one of said sequences comprises the sequence of SEQ ID NO. 2, 4, or 6; (e) a data set representing a polynucleotide sequence comprising the sequence of SEQ ID NO. 1, 3, or 5; and (f) a data set representing a polynucleotide sequence encoding a polypeptide sequence comprising the sequence of SEQ ID NO. 2, 4, or 6.

A further embodiment of the invention provides a computer based method for performing homology identification, said method comprising the steps of providing a polynucleotide sequence comprising the sequence of SEQ ID NO. 1, 3, or 5 in a computer readable medium; and comparing said polynucleotide sequence to at least one polynucleotide or polypeptide sequence to identify homology.

A further embodiment of the invention provides a computer based method for performing homology identification, said method comprising the steps of: providing a polypeptide sequence comprising the sequence of SEQ ID NO. 2, 4, or 6 in a computer readable medium; and comparing said polypeptide sequence to at least one polynucleotide or polypeptide sequence to identify homology.

A further embodiment of the invention provides a computer based method for polynucleotide assembly, said method comprising the steps of: (a) providing a first polynucleotide sequence comprising the sequence of SEQ ID NO. 1, 3, or 5 in a computer readable medium; and (b) screening for at least one overlapping region between said first polynucleotide sequence and a second polynucleotide sequence.

A further embodiment of the invention provides a computer based method for performing homology identification, said method comprising the steps of: (a) providing a polynucleotide sequence comprising the sequence of SEQ ID NO. 1, 3, or 5 in a computer readable medium; and (b) comparing said polynucleotide sequence to at least one polynucleotide or polypeptide sequence to identify homology.

A further embodiment of the invention provides a computer based method for performing homology identification, said method comprising the steps of: (a) providing a polypeptide sequence comprising the sequence of SEQ ID NO. 2, 4, or 6 in a computer readable medium; and (b) comparing said polypeptide sequence to at least one polynucleotide or polypeptide sequence to identify homology.

A further embodiment of the invention provides a computer based method for polynucleotide assembly, said method comprising the steps of: (a) providing a first polynucleotide sequence comprising the sequence of SEQ ID NO. 1, 3, or 5 in a computer readable medium; and (b) screening for at least one overlapping region between said first polynucleotide sequence and a second polynucleotide sequence.

Various changes and modifications within the spirit and scope of the disclosed invention will become readily apparent to those skilled in the art from reading the following descriptions and from reading the other parts of the present disclosure.

### BRIEF DESCRIPTION OF THE DRAWINGS

The invention will now be described in relation to the drawings in which:

FIG. 1 shows a PCR amplification of H. pylori DNA. Panel A: PCR amplification using primers HPO479-F1 and HPO479-R1; Lane 1—250 bp ladder DNA size marker, Lane 2—HP0479 product from 26695, Lane 3—HP0479 product from strain 0:3 Lane 4—HP0479 product from Sydney strain, Lane 5—HP0479 product from Type strain (ATC43504), Lane 6—HP0479 product from strain PJ1. Expected size of the HP0479 PCR product using these primers was 1242 bp. Panel B: PCR amplification using primers HP0479-GF1 and HPO479-GR1. Lanes 1,8, and 17-250 bp ladder DNA size marker, Lane 2—H. pylori 26695, Lane 3—H. pylori 26695 HP0479 mutant 1, Lane 4—H. pylori 26695 HP0479 mutant 2, Lane 5—H. pylori Sydney strain, Lane 6—H. pylori Sydney strain BP0479 mutant 1, Lane 7—H. pylori Sydney strain HP0479 mutant 2, Lane 9—H. pylori strain PJ1, Lane 10—H. pylori strain PJ1 HP0479 mutant 1, Lane 11—H. pylori strain 0:3, Lane 12—H. pylori strain 0:3 BP0479 mutant 1, Lane 13—H. pylori strain ATCC 43504, Lane 14—H. pylori strain ATCC 43504 HP0479 mutant 1, Lane 15—H. pylori strain 0:1, Lane 16—H. pylori strain 0:1 HP0479 mutant.

FIG. 2 shows ClustalW multiple sequence alignment of HP0479 homologs from various H. pylori strains. HP0479S=HP0479 homolog in Sydney strain. JHP0431=HP0479 homolog in J99. BP0479P=HP0479 homolog in PJ1. HP04793=BP0479 homolog in 0:3. HP0479G=BP0479 from 26695.

FIG. 3 shows a blot of SDS-PAGE analysis of H. pylori whole cell LPS samples. Lane 1—H. pylori 26695 LPS, Lane 2—H. pylori 0479GM1 LPS, Lane 3—H. pylori Sydney strain LPS, Lane 4—H. pylori 0479SM1 LPS, Lane 5—H. pylori strain PJ1 LPS, Lane 6—H. pylori 0479PM1 LPS, Lane 7—H. pylori strain 0:3 LPS, Lane 8—H. pylori HP04793M1 LPS, Lane 9—H. pylori ATCC 43504 LPS, Lane 10—H. pylori HP0479TM1 LPS.

FIG. 4 shows blots of SDS-PAGE analysis of whole cell LPS samples from H. pylori mutant and parental strains. Lane 1—0:3 HP04793M1, Lane 2—0:3, Lane 3—26695 HP0479GM1, Lane 4—26695, Lane 5—Sydney HP0479SM1, Lane 6—Sydney, Lane 7—PJ1 HP0479PM1, Lane 8—PJ1. Panel A show LPS silver stained. Panel B shows LPS immunoblotted with mouse monoclonal antibody raised against Lewis X. Panel C shows LPS immunoblotted with mouse monoclonal antibody raised against Lewis Y. Panel D shows LPS immunoblotted with rabbit polyclonal antibody raised against PJ1. The arrow in panel B shows the position of a faint band in lane 2.

FIG. 5 shows a blot of a Silver stain of an SDS-PAGE gel of H. pylori Sydney strain and H. pylori 0480M1 whole cell LPS samples. Lane 1—H. pylori Sydney strain LPS, Lane 2—H. pylori 0480SM1 LPS.

FIG. 6 CZE-ES-MS and CZE-MS-MS (+ion mode) analysis of 0.1M sodium acetate buffer treated LPS from H. pylori 0479 mutants. Separation conditions: 10 mM ammonium acetate containing 5% methanol, pH 9.0, +25 kV. (A) Extracted mass spectra at 14.6 min; (B) Tandem mass spectrum of precursor ions at m/z 1612; (C) Tandem mass spectrum of precursor ions at m/z 1392; (D) Tandem mass spectrum of precursor ions at m/z 1271; (E) Tandem mass spectrum of precursor ions at m/z 1246. Separation conditions as in (A) except nitrogen collision gas; E_{lab}: 60 eV (laboratory frame of reference).

FIG. 7 shows the structure of H. pylori HP0479 mutant and parent LPS from strains 26695 and SS1.

FIG. 8 shows a flow cytometric analysis of the adhesion of H. pylori SS1 strain and its mutants to Hutu 80 cells.

FIG. 9 shows the molecular structure of Helicobacter pylori LPS.

### DETAILED DESCRIPTION OF THE INVENTION

In accordance with the present invention there may be employed conventional molecular biology, microbiology, and recombinant DNA techniques within the skill of the art. Such techniques are explained fully in the literature. See for example, Sambrook, Fritsch, & Maniatis, Molecular Cloning: A Laboratory Manual, Second Edition (1989) Cold Spring Harbor Laboratory Press, Cold Spring Harbor, N.Y.); DNA Cloning: A Practical Approach, Volumes I and II (D. N. Glover ed. 1985); Oligonucleotide Synthesis (M. J. Gait ed. 1984); Nucleic Acid Hybridization B. D. Hames & S. J. Higgins eds. (1985); Transcription and Translation B. D. Hames & S. J. Higgins eds (1984); Animal Cell Culture R. I. Freshney, ed. (1986); Immobilized Cells and enzymes IRL Press, (1986); and B. Perbal, A Practical Guide to Molecular Cloning (1984).

Glossary

The following definitions are provided to facilitate understanding of certain terms used herein.

The term “complementary” refers to the natural binding of polynucleotides under permissive salt and temperature conditions by base-pairing. For example, the sequence “A-G-T” binds to the complementary sequence “T-C-A”. Complementarity between two single-stranded molecules may be “partial”, in which only some of the nucleic acids bind, or it may be complete when total complementarity exists between the single stranded molecules.

The term “consisting essentially of” or “consisting of” a polynucleotide sequence refers to the disclosed polynucleotide sequence, and also encompasses polynucleotide sequences which are identical except for a base change or substitution therein. As known to those skilled in the art, a limited number of base changes or substitutions may be made in a short oligonucleotide sequence resulting in a sequence maintaining substantial function (ranging from approximately 50% to greater than 100% of the activity) of the original unmodified sequence.

“Disease(s)” means a condition or disease caused by or related to infection by a bacteria that comprises a polypeptide or polynucleotide of the invention.

“Host cell” is a cell which has been transformed or transfected, or is capable of being transformed or transfected by an exogenous polynucleotide sequence.

“Identity,” as known in the art, is a relationship between two or more polypeptide sequences or two or more polynucleotide sequences, as determined by comparing the sequences. “Identity” also refers to the degree of sequence relatedness between polypeptide or polynucleotide sequences as determined by the match between strings of such sequences. “Identity” may be calculated by conventional methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48. 1073 (1988). Methods to determine identity are designed to give the highest match between the sequences tested. Methods to determine identity are codified in publicly available computer programs. Examples of computer program methods to determine identity between two sequences include, but are not limited to, the GCG program package (Devereux, J., et al., Nucleic Acids Research 12(1). 387 (1984)), BLASTP, BLASTN, and FASTA (Atschul, S. F. et al., J. Molec. Biol 215: 403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990). The Smith Waterman algorithm known in the art may also be used to determine identity.

Parameters for comparison of polypeptide sequences include the following: (1) Algorithm: Needleman and Wunsch, J. Mol Biol. 48: 443-453 (1970); (2) Comparison matrix: BLOSSUM62 from Hentikoff and Hentikoff, Proc. Natl. Acad. Sci. USA. 89:10915-10919 (1992); (3) Gap Penalty: 12; and (4) Gap Length Penalty: 4. A useful publicly available program with these parameters is the “gap” program from Genetics Computer Group, Madison Wis. The above-mentioned comparison parameters are the default parameters for peptide comparisons (along with no penalty for end gaps).

Parameters for comparison of polynucleotide sequences include the following: (1) Algorithm: Needleman and Wunsch, J. Mol Biol. 48: 443-453 (1970); (2) Comparison matrix: matches=+10, mismatch=0; (3) Gap Penalty: 50; and (4) Gap Length Penalty: 3. The “gap” program from Genetics Computer Group, Madison, Wis. is a publicly available program with these default parameters for nucleic acid comparisons.

A preferred meaning for “identity” for polynucleotides and polypeptides is as follows:

(1) Polynucleotide embodiments may include an isolated polynucleotide comprising a polynucleotide sequence having at least 50, 60, 70, 80, 85, 90, 95, 97 or 100% identity to the sequence of SEQ ID NO:1, 3, or 5, where the polynucleotide sequence may be identical to the sequence of SEQ ID NO: 1, 3, or 5 or may include up to a certain integer number of nucleotide alterations as compared to the sequence of SEQ ID NO: 1, 3, or 5. The alterations may be selected from the group consisting of at least one nucleotide deletion, substitution, including transition and transversion, or insertion. The alterations may occur at the 5′ or 3′ terminal positions of the sequence of SEQ ID NO: 1, 3, or 5 or anywhere between those terminal positions, interspersed either individually among the nucleotides in the sequence of SEQ ID NO: 1, 3, or 5 or in one or more contiguous groups within this sequence. The number of nucleotide alterations can be determined by multiplying the total number of nucleotides in SEQ ID NO:1, 3, or 5 by the integer defining the percent identity divided by 100 and then subtracting that product from the total number of nucleotides in SEQ ID NO:1, 3, or 5.

(2) Polypeptide embodiments may include an isolated polypeptide comprising a polypeptide having at least a 50,60, 70, 80, 85, 90, 95, 97 or 100% identity to a polypeptide sequence of SEQ ID NO:2, 4, or 6 where the polypeptide sequence may be identical to the sequence of SEQ ID NO: 2, 4, or 6 or may include up to a certain integer number of amino acid alterations as compared to the sequence. The alterations may be selected from the group consisting of at least one amino acid deletion, substitution, including conservative and non-conservative substitution, or insertion, and where the alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations is determined by multiplying the total number of amino acids in SEQ ID NO: 2, 4, or 6 by the integer defining the percent identity divided by 100 and then subtracting that product from said total number of amino acids in SEQ ID NO: 2,4, or 6.

The term “isolated” refers to a polynucleotide or polypeptide changed and/or removed from its natural environment, purified or separated, or substantially free of cellular material or culture medium when produced by recombinant DNA techniques, or chemical reactants, or other chemicals when chemically synthesized. A polynucleotide or polypeptide that is introduced into an organism by transformation, genetic manipulation, or any other recombinant method is “isolated” even if it is still present in an organism, which may be living or non-living. Preferably, an isolated polynucleotide or polypeptide is at least 60% free, more preferably at least 75% free, and most preferably at least 90% free from other components with which they are naturally associated.

“Polynucleotide(s)” generally refers to any polyribonucleotide or polydeoxribonucleotide, which may be unmodified RNA or DNA or modified RNA or DNA, including mRNAs, DNAs, cDNAs and genomic DNA. “Polynucleotide(s)” include, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. The term also includes triple-stranded regions comprising RNA or DNA or both RNA and DNA. The strands in such triple-stranded regions may be from the same molecule or from different molecules. The regions may include all or one or more of the molecules, but typically involve only a region of some of the molecules. One of the molecules of a triple-helical region often is an oligonucleotide. As used herein, the term “polynucleotide(s)” also includes DNAs or RNAs as described herein that contain one or more modified bases. Thus, DNAs or RNAs with backbones modified for stability or for other reasons are within the meaning of the term “polynucleotide(s)”. “Polynucleotide(s)” also includes DNAs or RNAs comprising unusual bases, such as inosine, or modified bases, such as tritylated bases, to name just two examples. A great variety of modifications have been made to DNA and RNA that serve many useful purposes known to those of skill in the art and the term “polynucleotide(s)” embraces such chemically, enzymatically or metabolically modified forms of polynucleotides, as well as the chemical forms of DNA and RNA characteristic of viruses and cells, including, for example, simple and complex cells. “Polynucleotide(s)” also includes short polynucleotides often referred to as oligonucleotide(s). The term “polynucleotides” and in particular DNA or RNA, refers only to the primary and secondary structure and it does not limit it to any particular tertiary forms.

The term “polynucleotide encoding a polypeptide” encompasses polynucleotides that include a sequence encoding a polypeptide of the invention, particularly a bacteria polypeptide and more particularly a polypeptide of Helicobacter pylori having an amino acid sequence set out in SEQ ID NO: 2, 4, or 6. The term also contemplates polynucleotides that include a single continuous region or discontinuous regions encoding the polypeptide (e.g. interrupted by integrated phage or an insertion sequence or editing) together with additional regions, that also may contain coding and/or non-coding sequences.

“Polypeptide(s)” refers to any peptide or protein comprising two or more amino acids joined to each other by peptide bonds or modified peptide bonds. The term includes both short chains, commonly referred to as peptides, oligopeptides and oligomers and to longer chains generally referred to as proteins. Polypeptides may contain amino acids other than the 20 gene encoded amino acids. “Polypeptide(s)” as used herein includes those modified either by natural processes, such as processing and other post-translational modifications, but also by chemical modification techniques. Such modifications are well described in basic texts and research literature, and they are well known to those of skill in the art. The same type of modification may be present in the same or varying degree at several sites in a given polypeptide, and a given polypeptide may contain many types of modifications. Modifications may occur anywhere in a polypeptide, including the peptide backbone, the amino acid side-chains, and the amino or carboxyl termini. Examples of modifications include, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, glycosylation, lipid attachment, sulfation, gamma-carboxylation of glutamic acid residues, hydroxylation and ADP-ribosylation, selenoylation, sulfation, transfer-RNA mediated addition of amino acids to proteins, such as arginylation, and ubiquitination. (See, for example, PROTEINS—STRUCTURE AND MOLECULAR PROPERTIES, 2^{nd} Ed., T. E. Creighton, W. H. Freeman and Company, New York (1993) and Wold, F., Posttranslational Protein Modifications: Perspectives and Prospects, pgs. 1-12 in POSTRRANSLATIONAL COVALENT MODIFICATION OF PROTEINS, B. C. Johnson, Ed., Academic Press, New York (1983); Seifter et al., Meth. Enzymol. 182:626-646 (1990) and Rattan et al., Protein Synthesis: Posttranslational Modifications and Aging, Ann. N.Y. Acad. Sci. 663: 48-62 (1992). “Polypeptides” may be branched or cyclic, with or without branching. These polypeptides may result from post-translational natural processes and may be made by entirely synthetic methods.

“Variant(s)” as used herein refers to a polynucleotide or polypeptide that differs from a reference polynucleotide or polypeptide respectively, but retains essential properties. A typical variant of a polynucleotide differs in nucleotide sequence from another polynucleotide. Changes in the nucleotide sequence of the variant may or may not alter the amino acid sequence of an encoded polypeptide. Nucleotide changes may result in amino acid substitutions, additions, deletions, fusions and truncations in the polypeptide encoded by the reference sequence. A typical variant of a polypeptide differs in amino acid sequence from another, reference polypeptide. Differences are generally limited so that the sequences of the reference polypeptide and the variant are very similar overall and, in many regions, identical. A variant may differ in amino acid sequence by one or more substitutions, additions, deletions in any combination. A substituted or inserted amino acid residue may or may not be one encoded by the genetic code. A variant of a polynucleotide or polypeptide may be a naturally occurring variant such as an allelic variant, or it may be a variant that is not known to occur naturally. Mutagenesis techniques, direct synthesis, and other recombinant methods known to skilled artisans may be used to produce non-naturally occurring variants of polynucleotides and polypeptides.

A “ligand” refers to a compound or entity that associates with a polypeptide of the invention or part thereof, including acceptor molecules or analogues or parts thereof, and donor molecules or analogues or parts thereof.

A “donor molecule” refers to a molecule capable of donating a sugar to an acceptor molecule, via the action of a DDHepT polypeptide. The donor molecule may be di- or poly-saccharides, sugar 1-phosphates, or, most commonly, nucleotide diphosphosugars (ADP-sugars), or nucleotide phosphosugars. In a preferred embodiment, the donor molecule is ADP-mannoheptose.

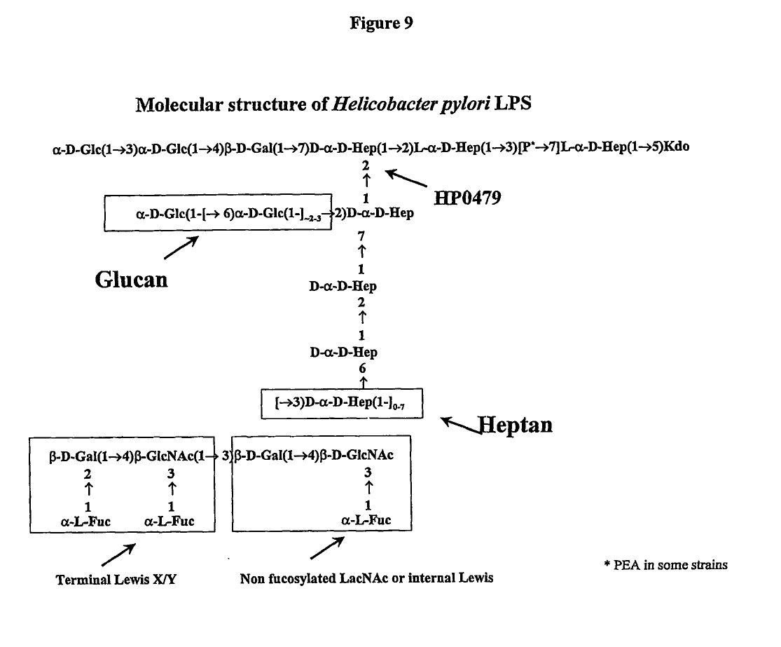

An acceptor molecule is capable of accepting a sugar from a donor molecule, via the action of a DDHepT polypeptide. It may, for example, comprise a terminal sugar residue for transfer purposes. The acceptor molecule or aglycone can be, for example, a lipid, a protein, a heterocyclic compound, an antibiotic, a peptide, an amino acid, an aromatic or aliphatic alcohol or thiol or another carbohydrate residue. In a preferred embodiment, the acceptor molecule is or comprises a terminal D-α-D-heptose. (See FIG. 9 for the LPS structure).

An analogue of a donor or acceptor molecule is one which mimics the donor or acceptor molecule binding to a DDHepT polypeptide but which is incapable (or has a significantly reduced capacity) to take part in the transfer reaction.

Polynucleotides

As hereinbefore mentioned, the invention provides isolated polynucleotides, (including a full length DDhepT gene) that encode DDHepT polypeptides, or fragments, variants, homologs thereof, and polynucleotides having substantial identity thereto, and variants thereof. Preferably, the polynucleotides encode polypeptides that retain substantially the same biological function or activity of a mature DDHepT.

In an embodiment of the invention an isolated polynucleotide is contemplated which comprises:

(i) a polynucleotide encoding a polypeptide having substantial sequence identity, preferably at least 50%, more preferably at least 70% sequence identity, with an amino acid sequence of SEQ. ID. NO. 2, 4, or 6;

(ii) polynucleotides complementary to (i);

(iii) polynucleotides differing from any of the polynucleotides of (i) or (ii) in codon sequences due to the degeneracy of the genetic code;

(iv) a polynucleotide comprising at least 10, 15, or 18, preferably at least 20 nucleotides and capable of hybridizing under stringent conditions to a polynucleotide of SEQ. ID. NO. 1, 3, or 5 or to a degenerate form thereof;

(v) a polynucleotide encoding an allelic or species variation of a polypeptide comprising an amino acid sequence of SEQ. ID. NO. 2, 4, or 6; or

(vi) a fragment, or allelic or species variation of (i), (ii) or (iii)

In a specific embodiment, the isolated polynucleotide comprises:

(i) a polynucleotide having substantial sequence identity, preferably at least 50%, more preferably at least 70% sequence identity with a sequence of SEQ. ID. NO. 1, 3, or 5;

(ii) polynucleotides complementary to (i), preferably complementary to a full sequence of SEQ. ID. NO. 1, 3, or 5;

(iii) polynucleotides differing from any of the nucleic acids of (i) to (ii) in codon sequences due to the degeneracy of the genetic code; or

(iv) a fragment, or allelic or species variation of (i), (ii) or (iii).

In a preferred embodiment the isolated nucleic acid comprises a polynucleotide encoded by an amino acid sequence of SEQ. ID. NO. 2, 4, or 6 or comprises or consists essentially of a polynucleotide of SEQ. ID. NO. 1, 3, or 5 wherein T can also be U. The DNA sequence set out in SEQ ID NO: 1, 3, or 5 contains an open reading frame encoding a polypeptide comprising the amino acid residues set forth in SEQ ID NO: 2, 4, or 6, respectively, with a deduced molecular weight that can be calculated using amino acid residue molecular weight values well known in the art.

Preferably, a polynucleotide of the present invention has substantial sequence identity using the preferred computer programs cited herein, for example at least 50%, 60%, 70%, 75%, 80%, 90%, more preferably at least 95%, 96%, 97%, 98%, or 99% sequence identity to a sequence of SEQ. ID. NO. 1, 3, or 5.

Isolated nucleic acid molecules encoding a polypeptide of the invention and having a sequence which differs from a polynucleotide of SEQ. ID. NO. 1, 3, or 5 due to degeneracy in the genetic code are also within the scope of the invention. As one example, DNA sequence variations within DDhepT may result in silent mutations which do not affect the amino acid sequence. Variations in one or more nucleotides may exist among organisms within a genus due to natural allelic variation. Any and all such nucleic acid variations are within the scope of the invention. DNA sequence variations may also occur which lead to changes in the amino acid sequence of a polypeptide of the invention. These amino acid variations are also within the scope of the present invention. In addition, species variations i.e. variations in nucleotide sequence naturally occurring among different species, are within the scope of the invention.

The invention contemplates the coding sequence for the mature polypeptide or a fragment thereof, by itself as well as the coding sequence for the mature polypeptide or a fragment in reading frame with other coding sequences, including those encoding a leader or secretory sequence, a pre, or pro- or prepro-protein sequence. A polynucleotide of the invention may also contain non-coding sequences, including, but not limited to non-coding 5′ and 3′ sequences, such as the transcribed, non-translated sequences, termination signals, ribosome binding sites, sequences that stabilize mRNA, introns, polyadenylation signals, and additional coding sequence which encode additional amino acids. The additional sequences may be a marker sequence that facilitates purification of the fused polypeptide, the sequences may play a role in processing of a polypeptide from precursor to a mature form, may allow protein transport, may lengthen or shorten protein half-life or may facilitate manipulation of a protein for assay or production. Additional sequences may be at the amino or carboxyl-terminal end or interior to the mature polypeptide.

Polynucleotides of the invention also include, but are not limited to, polynucleotides comprising a structural gene and its naturally associated sequences that control gene expression.

Also included in the invention are polynucleotides of the formula:

X—(R₁)ₘ(Z)-(R₂)ₙ—Y

wherein, at the 5′ end of the molecule, X is hydrogen or a metal or together with Y defines a covalent bond, and at the 3′ end of the molecule, Y is hydrogen or a metal or together with X defines a covalent bond, each occurrence of R₁ and R₂ is independently any nucleic acid residue, m is an integer between 1 and 3000 or zero, preferably between 1 and 1000, n is an integer between 1 and 3000 or zero, preferably between 1 and 1000, and Z is a polynucleotide sequence of the invention, particularly a sequence selected from SEQ ID NO: 1, 3, or 5. Any stretch of nucleotide residues denoted by either R group, where m and/or n is greater than 1, may be either a heteropolymer or a homopolymer, preferably a heteropolymer. In an embodiment, X and Y together define a covalent bond and the polynucleotide of the above formula is a closed, circular polynucleotide, which can be a double-stranded polynucleotide wherein the formula shows a first strand to which the second strand is complementary.

Fragments of a polynucleotide of the invention, include fragments that are a stretch of at least about 10, 15, 18, 20, 40, 50, 100, or 150 nucleotides, more typically at least 50 to 100 nucleotides but less than 2 kb. It will further be appreciated that variant forms of the polynucleotides of the invention which arise by alternative splicing of an mRNA corresponding to a cDNA of the invention are encompassed by the invention. Polynucleotides that encode for variants of polypeptides of the invention are particularly contemplated that have an amino acid sequence of SEQ ID NO: 2, 4, or 6, in which several, a few, 5 to 10, 1 to 5, 1 to 3, 2, 1, or no amino acid residues are substituted. Preferred among these variants are silent substitutions, additions, and deletions that do not alter the properties and activities of the polypeptide.

Another aspect of the invention provides a polynucleotide which hybridizes under selective conditions, e.g. high stringency conditions, to a polynucleotide which comprises a sequence which encodes a polypeptide of the invention. Preferably the sequence encodes an amino acid sequence of SEQ. ID. NO. 2, 4, or 6 or part thereof and comprises at least 18 nucleotides. Selectivity of hybridization occurs with a certain degree of specificity rather than being random. Appropriate stringency conditions which promote DNA hybridization are known to those skilled in the art, or can be found in Current Protocols in Molecular Biology, John Wiley & Sons, N.Y. (1989), 6.3.1-6.3.6. For example, hybridization may occur at 30° C. in 750 mM NaCl, 75 mM trisodium citrate, and 1% SDS, preferably 37° C. in 500 mM NaCl, 500 mM trisodium citrate, 1% SDS, 35% formamide, and 100 g/ml denatured salmon sperm DNA (ssDNA), and more preferably 42° C. in 250 mM NaCl, 25 mM trisodium citrate, 1% SDS, 50% formamide, and 200 μg/ml ssDNA. Useful variations on these conditions will be readily apparent to those skilled in the art.

The stringency may be selected based on the conditions used in the wash step. Wash step stringency conditions may be defined by salt concentration and by temperature. Generally, wash stringency can be increased by decreasing salt concentration or by increasing temperature. By way of example, a stringent salt concentration for the wash step is preferably less than about 30 mM NaCl and 3 mM trisodium citrate, and more preferably less than about 15 mM NaCl and 1.5 mM trisodium citrate. Stringent temperature conditions will generally include temperatures of a least about 25° C., more preferably at least about 68° C. In a preferred embodiment, the wash steps will be carried out at 42° C. in 15 mM NaCl, 1.5 mM trisodium citrate, and 0.1% SDS. In a more preferred embodiment the wash steps are carried out at 68° C. in 15 mM NaCl, 1.5 mM trisodium citrate, and 0.1% SDS. Variations on these conditions will be readily apparent to those skilled in the art.

The polynucleotides of the inventions are preferably derived from Helicobacter pylori, however, they may be obtained from organisms of the same taxonomic genus. They may also be obtained from organisms of the same taxonomic family or order.

An isolated polynucleotide of the invention which comprises DNA can be isolated by preparing a labeled nucleic acid probe based on all or part of a nucleic acid sequence of SEQ. ID. NO. 1, 3, or 5. The labeled nucleic acid probe is used to screen an appropriate DNA library (e.g. a cDNA or genomic DNA library). For example, a cDNA library can be used to isolate a cDNA encoding a polypeptide of the invention by screening the library with the labeled probe using standard techniques. Alternatively, a genomic DNA library can be similarly screened to isolate a genomic clone encompassing a DDhepT gene. Polynucleotides isolated by screening of a cDNA or genomic DNA library can be sequenced by standard techniques.

An isolated polynucleotide of the invention which is DNA can also be isolated by selectively amplifying a polynucleotide of the invention. “Amplifying” or “amplification” refers to the production of additional copies of a nucleic acid sequence and is generally carried out using polymerase chain reaction (PCR) technologies well known in the art (Dieffenbach, C. W. and G. S. Dveksler (1995) PCR Primer, a Laboratory Manual, Cold Spring Harbor Press, Plainview, N.Y.). In particular, it is possible to design synthetic oligonucleotide primers from a nucleotide sequence of SEQ. ID. NO. 1, 3, or 5 for use in PCR. Examples of suitable primers are the sequences of SEQ ID NO. 7 through 14. A nucleic acid can be amplified from cDNA or genomic DNA using these oligonucleotide primers and standard PCR amplification techniques. The nucleic acid so amplified can be cloned into an appropriate vector and characterized by DNA sequence analysis. cDNA may be prepared from mRNA, by isolating total cellular mRNA by a variety of techniques, for example, by using the guanidinium-thiocyanate extraction procedure of Chirgwin et al., Biochemistry, 18, 5294-5299 (1979). cDNA is then synthesized from the mRNA using reverse transcriptase (for example, Moloney MLV reverse transcriptase available from Gibco/BRL, Bethesda, Md., or AMV reverse transcriptase available from Seikagaku America, Inc., St. Petersburg, Fla.).

An isolated polynucleotide of the invention which is RNA can be isolated by cloning a cDNA encoding a polypeptide of the invention into an appropriate vector which allows for transcription of the cDNA to produce an RNA molecule which encodes the polypeptide. For example, a cDNA can be cloned downstream of a bacteriophage promoter, (e.g. a T7 promoter) in a vector, cDNA can be transcribed in vitro with T7 polymerase, and the resultant RNA can be isolated by conventional techniques.

A polynucleotide of the invention may be engineered using methods known in the art to alter the DDhepT encoding sequence for a variety of purposes including modification of the cloning, processing, and/or expression of the gene product. Procedures such as DNA shuffling by random fragmentation and PCR reassembly of gene fragments and synthetic oligonucleotides may be used to engineer the nucleic acid molecules. Mutations may be introduced by oligonucleotide-mediated site-directed mutagenesis to create for example new restriction sites, change codon preference, or produce variants.

Polynucleotides of the invention may be chemically synthesized using standard techniques. Methods of chemically synthesizing polydeoxynucleotides are known, including but not limited to solid-phase synthesis which, like peptide synthesis, has been fully automated in commercially available DNA synthesizers (See e.g., Itakura et al. U.S. Pat. No. 4,598,049; Caruthers et al. U.S. Pat. No. 4,458,066; and Itakura U.S. Pat. Nos. 4,401,796 and 4,373,071).

Determination of whether a particular polynucleotide is a DDhepT gene or encodes a polypeptide of the invention can be accomplished by expressing the cDNA in an appropriate host cell by standard techniques, and testing the expressed protein in the methods described herein. A cDNA encoding a polypeptide of the invention can be sequenced by standard techniques, such as dideoxynucleotide chain termination or Maxam-Gilbert chemical sequencing, to determine the nucleic acid sequence and the predicted amino acid sequence of the encoded protein.

The polynucleotides of the invention may be extended using a partial nucleotide sequence and various PCR-based methods known in the art to detect upstream sequences such as promoters and regulatory elements. For example, restriction-site PCR which uses universal and nested primers to amplify unknown sequences from genomic DNA within a cloning vector may be employed (See Sarkar, G, PCR Methods Applic. 2:318-322, 1993). Inverse PCR which uses primers that extend in divergent directions to amplify unknown sequences from a circularized template may also be used. The template in inverse PCR is derived from restriction fragments adjacent to known sequences in human and yeast artificial chromosome DNA (See e.g. Lagerstrom, M., at al, PCR Methods Applic. 1:111-119, 1991). Other methods for retrieving unknown sequences are known in the art (e.g. Parker, J. D. et al, Nucleic Acids Res. 19:305-306, 1991). In addition, PCR, nested primers, and PROMOTERFINDER libraries (Clontech, Palo Alto, Calif.) may be used to walk genomic DNA.

It is preferable when screening for full-length cDNAs to use libraries that have been size-selected to include larger cDNAs. For situations in which an oligo d(T) library does not yield a full-length cDNA, it is preferable to use random-primed libraries which often include sequences containing the 5′ regions of genes. Genomic libraries may be useful for extending the sequence into 5′non-translated regulatory regions.

Commercially available capillary electrophoresis systems may be employed to analyse the size or confirm the sequence of PCR or sequencing products. The system may use flowable polymers for electrophoretic separation, four different nucleotide-specific, laser-stimulated fluorescent dyes, and a charge coupled device camera for detection of the emitted wavelengths. Commercially available software (e.g. GENOTYPER and SEQUENCE NAVIGATOR, Perkin-Elmer) may convert the output/light intensity to electrical signal, and the entire process from loading of samples, and computer analysis and electronic data display may be computer controlled. This procedure may be particularly useful for sequencing small DNA fragments which may be present in limited amounts in a particular sample.

In accordance with another aspect of the invention, the polynucleotides isolated using the methods described herein are mutant DDhepT gene alleles. For example, the mutant alleles may be isolated from organisms either known or proposed to contribute to a disease. Mutant alleles and mutant allele products may be used in therapeutic and diagnostic methods described herein. For example, a cDNA of a mutant DDhepT gene may be isolated using PCR as described herein, and the DNA sequence of the mutant allele may be compared to the normal allele to ascertain the mutation(s) responsible for the loss or alteration of function of the mutant gene product. A genomic library can also be constructed using DNA from an organism suspected of or known to carry a mutant allele, or a cDNA library can be constructed using RNA from organisms known to express the mutant allele. A polynucleotide encoding a normal DDhepT gene or any suitable fragment thereof, may then be labeled and used as a probe to identify the corresponding mutant allele in such libraries. Clones containing mutant sequences can be purified and subjected to sequence analysis. In addition, an expression library can be constructed using cDNA from RNA isolated from organisms known or suspected to express a mutant DDhepT allele. Gene products from putatively mutant organisms may be expressed and screened, for example using antibodies specific for a polypeptide as described herein. Library clones identified using the antibodies can be purified and subjected to sequence analysis.

Antisense molecules and ribozymes are contemplated within the scope of the invention. “Antisense” refers to any composition containing nucleotide sequences which are complementary to a specific DNA or RNA sequence. Ribozymes are enzymatic RNA molecules that can be used to catalyze the specific cleavage of RNA. Antisense molecules and ribozymes may be prepared by any method known in the art for the synthesis of polynucleotides. These include techniques for chemically synthesizing oligonucleotides such as solid phase phosphoramidite chemical synthesis. Alternatively, RNA molecules may be generated by in vitro and in vivo transcription of DNA sequences encoding a polypeptide of the invention. Such DNA sequences may be incorporated into a wide variety of vectors with suitable RNA polymerase promoters such as 17 or SP6. Alternatively, these cDNA constructs that synthesize antisense RNA constitutively or inducibly can be introduced into organisms. RNA molecules may be modified to increase intracellular stability and half-life. Possible modifications include, but are not limited to, the addition of flanking sequences at the 5′ and/or 3′ ends of the molecule or the use of phosphorothioate or 2′O-methyl rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

Polypeptides

A polypeptide of the invention includes a polypeptide of SEQ.ID. NO: 2, 4, or 6, particularly those which have the biological activity of a DDHepT. In addition to polypeptides comprising an amino acid sequence of SEQ.ID. NO. 2, 4, or 6 the polypeptides of the present invention include truncations or fragments, and variants, and homologs.

Truncated polypeptides may comprise peptides of between 3 and 70 amino acid residues, ranging in size from a tripeptide to a 50 mer polypeptide, preferably 30 to 50 amino acids. In one aspect of the invention, fragments of a polypeptide of the invention are provided having an amino acid sequence of at least five consecutive amino acids of SEQ.ID. NO. 2, 4, or 6 where no amino acid sequence of five or more, six or more, seven or more, or eight or more, consecutive amino acids present in the fragment is present in a polypeptide other than a DDHepT of the invention. In an embodiment of the invention the fragment is a stretch of amino acid residues of at least 12 to 20 contiguous amino acids from particular sequences such as the sequences of SEQ.ID. NO. 2, 4, or 6. The fragments may be immunogenic and preferably are not immunoreactive with antibodies that are immunoreactive to polypeptides other than a DDHepT of the invention. Particularly preferred are fragments that are antigenic or immunogenic in an animal, especially in a human.

A fragment may be characterized by structural or functional attributes such as fragments that comprise alpha-helix and alpha-helix forming regions, beta-sheet and beta-sheet-forming regions, turn and turn-forming regions, coil and coil-forming regions, hydrophilic regions, hydrophobic regions, alpha amphipathic regions, beta amphipathic regions, flexible regions, surface-forming regions, substrate binding regions, and high antigenic index regions.

In a preferred embodiment, the invention provides biologically active fragments which are those fragments that mediate activities of a DDHepT, including those with a similar activity or an improved activity, or with a decreased undesirable activity. Particularly preferred are fragments comprising domains of enzymes that confer a function essential for viability of Helicobacter species or the ability to initiate, maintain, or cause disease in an individual, particularly a human.

Truncated polypeptides may have an amino group (—NH2), a hydrophobic group (for example, carbobenzoxyl, dansyl, or T-butyloxycarbonyl), an acetyl group, a 9-fluorenylmethoxy-carbonyl (PMOC) group, or a macromolecule including but not limited to lipid-fatty acid conjugates, polyethylene glycol, or carbohydrates at the amino terminal end. The truncated polypeptides may have a carboxyl group, an amido group, a T-butyloxycarbonyl group, or a macromolecule including but not limited to lipid-fatty acid conjugates, polyethylene glycol, or carbohydrates at the carboxy terminal end.

A truncated polypeptide or fragment may be “free-standing,” or comprised within a larger polypeptide of which they form a part or region, most preferably as a single continuous region, of a single larger polypeptide.

The polypeptides of the invention may also include variants of a DDHepT of the invention, and/or truncations thereof as described herein, which may include, but are not limited to a polypeptide of the invention containing one or more amino acid substitutions, insertions, and/or deletions. Amino acid substitutions may be of a conserved or non-conserved nature. Conserved amino acid substitutions involve replacing one or more amino acids of a DDHepT amino acid sequence with amino acids of similar charge, size, and/or hydrophobicity characteristics. When only conserved substitutions are made the resulting analog is preferably functionally equivalent to a DDHepT of the invention. Non-conserved substitutions involve replacing one or more amino acids of the DDHepT amino acid sequence with one or more amino acids which possess dissimilar charge, size, and/or hydrophobicity characteristics.

One or more amino acid insertions may be introduced into a polypeptide of the invention. Amino acid insertions may consist of single amino acid residues or sequential amino acids ranging from 2 to 15 amino acids in length.

Deletions may consist of the removal of one or more amino acids, or discrete portions from the DDHepT amino acid sequence. The deleted amino acids may or may not be contiguous. The lower limit length of the resulting analog with a deletion mutation is about 10 amino acids, preferably 50 amino acids.

Allelic variants of a DDHepT at the protein level differ from one another by only one, or at most, a few amino acid substitutions. A species variation of a DDHepT polypeptide is a variation which is naturally occurring among different species of an organism.

The polypeptides of the invention include homologs of a DDHepT and/or truncations thereof as described herein. Such DDHepT homologs include proteins whose amino acid sequences are comprised of the amino acid sequences of DDHepT regions from other species that hybridize under selective hybridization conditions (see discussion of selective and in particular stringent hybridization conditions herein) with a probe used to obtain a polypeptide. These homologs will generally have the same regions which are characteristic of a DDHepT polypeptide. It is anticipated that a protein comprising an amino acid sequence which has at least 50%, 60%, 70%, 75%, 80%, 85%, or 90% identity, more preferably 95%, 96%, 97%, 98%, or 99% identity with an amino acid sequence of SEQ. ID. NO. 2, 4, or 6 will be a homolog of a polypeptide of the invention. A percent amino acid sequence homology or identity is calculated using the methods described herein, preferably the computer programs described herein.

The invention also contemplates isoforms of polypeptides of the invention. An isoform contains the same number and kinds of amino acids as a polypeptide of the invention, but the isoform has a different molecular structure. The isoforms contemplated by the present invention preferably have the same properties as a polypeptide of the invention as described herein.

The present invention also provides a polypeptide of the invention conjugated with a selected protein, or a marker or other glycosyltransferase, to produce fusion proteins or chimeric proteins.

Also included in the invention are polypeptides of the formula:

X—(R₁)ₘ-(Z)-(R₂)ₙ—Y

wherein, at the amino terminus, X is hydrogen or a metal, and at the carboxy terminus Y is hydrogen or a metal, or together Y and X define a covalent bond, each occurrence of R₁ and R₂ is independently any amino acid residue, m is an integer between 1 and 1000 or zero, preferably between 1 and 1000, n is an integer between 1 and 3000 or zero, preferably between 1 and 1000, and Z is a polypeptide of the invention, particularly a sequence selected from SEQ ID NO: 2, 4, or 6. Any stretch of amino acid residues denoted by either R group, where m and/or n is greater than 1, may be either a heteropolymer or a homopolymer, preferably a heteropolymer. Where, in a preferred embodiment, X and Y together define a covalent bond, the polypeptide of the above formula is a closed, circular polypeptide.

A polypeptide of the invention may be prepared using recombinant DNA methods. Accordingly, polynucleotides of the present invention having a sequence which encodes a polypeptide of the invention may be incorporated in a known manner into an appropriate expression vector which ensures good expression of the polypeptide. Possible expression vectors include but are not limited to chromosomal, episomal and virus-derived vectors, so long as the vector is compatible with the host cell used. Representative examples of vectors are vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids

The invention therefore contemplates a recombinant expression vector comprising a polynucleotide of the invention, and the necessary regulatory sequences for the transcription and translation of the inserted sequence. Suitable regulatory sequences may be derived from a variety of sources, including bacterial, fungal, viral, mammalian, or insect genes (For example, see the regulatory sequences described in Goeddel, Gene Expression Technology: Methods in Enzymology 185, Academic Press, San Diego, Calif. (1990). Selection of appropriate regulatory sequences is dependent on the host cell chosen as discussed below, and may be readily accomplished by one of ordinary skill in the art. The necessary regulatory sequences may be supplied by the native polypeptide and/or its flanking regions.

The invention further provides a recombinant expression vector comprising a polynucleotide of the invention cloned into the expression vector in an antisense orientation. That is, the DNA molecule is linked to a regulatory sequence in a manner which allows for expression, by transcription of the DNA molecule, of an RNA molecule which is antisense to a polynucleotide sequence of SEQ. ID. NO. 1, 3, or 5. Regulatory sequences linked to the antisense nucleic acid can be chosen which direct the continuous expression of the antisense RNA molecule in a variety of cell types, for instance a viral promoter and/or enhancer, or regulatory sequences can be chosen which direct tissue or cell type specific expression of antisense RNA.

The recombinant expression vectors of the invention may also contain a marker gene which facilitates the selection of host cells transformed or transfected with a recombinant molecule of the invention. Examples of marker genes are genes encoding a protein such as G418, dhfr, npt, als, pat and hygromycin which confer resistance to certain drugs, β-galactosidase, chloramphenicol acetyltransferase, firefly luciferase, trpB, hisD, herpes simplex virus thymidine kinase, adenine phosphoribosyl transferase, or an immunoglobulin or portion thereof such as the Fc portion of an immunoglobulin preferably IgG. Visible markers such as anthocyanins, beta-glucuronidase and its substrate GUS, and luciferase and its substrate luciferin, can be used to identify transformants, and also to quantify the amount of transient or stable protein expression attributable to a specific vector system (Rhodes, C. et al. (1995) Mol. Biol. 55:121-131). The markers can be introduced on a separate vector from the nucleic acid of interest.

The recombinant expression vectors may also contain genes that encode a fusion moiety which provides increased expression of the recombinant polypeptide; increased solubility of the recombinant polypeptide; and aid in the purification of the target recombinant polypeptide by acting as a ligand in affinity purification. For example, a proteolytic cleavage site may be added to the target recombinant polypeptide to allow separation of the recombinant polypeptide from the fusion moiety subsequent to purification of the fusion protein. Typical fusion expression vectors include pGEX (Amrad Corp., Melbourne, Australia), pMAL (New England Biolabs, Beverly, Mass.) and pRIT5 (Pharmacia, Piscataway, N.J.) which fuse glutathione S-transferase (GST), maltose E binding protein, or protein A, respectively, to the recombinant protein.

The vectors may be introduced into host cells to produce a transformed or transfected host cell. The terms “transfected” and “transfection” encompass the introduction of nucleic acid (e.g. a vector) into a cell by one of many standard techniques. A cell is “transformed” by a nucleic acid when the transfected nucleic acid effects a phenotypic change. Prokaryotic cells can be transfected or transformed with nucleic acid by, for example, electroporation or calcium-chloride mediated transformation. Nucleic acid can be introduced into mammalian cells via conventional techniques such as calcium phosphate or calcium chloride co-precipitation, DEAE-dextran-mediated transfection, lipofectin, electroporation or microinjection. Suitable methods for transforming and transfecting host cells can be found in Sambrook et al. (Molecular Cloning: A Laboratory Manual, 2nd Edition, Cold Spring Harbor Laboratory press (1989)), and other laboratory textbooks.

Suitable host cells include a wide variety of prokaryotic and eukaryotic host cells. For example, the proteins of the invention may be expressed in bacterial cells such as E. coli, insect cells (using baculovirus), yeast cells, or mammalian cells. Other suitable host cells can be found in Goeddel, Gene Expression Technology: Methods in Enzymology 185, Academic Press, San Diego, Calif. (1991).

Examples of appropriate host cells include bacterial cells, such as Streptococci, Staphylococci, Enterococci, E. coli, Helicobacter, Streptomyces, and Bacillus subtilis cells; fungal cells, such as yeast cells and Aspergillus cells; insect cells such as Drosophila S2 and Spodoptera Sf9 cells; animal cells such as CHO, COS, HeLa, C127, 3T3, BHK, 293 and Bowes melanoma cells; and plant cells.

A host cell may also be chosen which modulates the expression of an inserted nucleic acid sequence, or modifies (e.g. glycosylation) and processes (e.g. cleaves) the polypeptide in a desired fashion. Host systems or cell lines may be selected which have specific and characteristic mechanisms for post-translational processing and modification of proteins. For long-term high-yield stable expression of the polypeptide, cell lines and host systems which stably express the gene product may be engineered.

Host cells and in particular cell lines produced using the methods described herein may be particularly useful in screening and evaluating compounds that modulate the activity of a polypeptide of the invention.

Polypeptides of the invention may also be prepared by chemical synthesis using techniques well known in the chemistry of proteins such as solid phase synthesis (Merrifield, 1964, J. Am. Chem. Assoc. 85:2149-2154) or synthesis in homogenous solution (Houbenweyl, 1987, Methods of Organic Chemistry, ed. E. Wansch, Vol. 15 I and II, Thieme, Stuttgart). Protein synthesis may be performed using manual procedures or by automation. Automated synthesis may be carried out, for example, using an Applied Biosystems 43 1A peptide synthesizer (Perkin Elmer). Various fragments of the polypeptides of the invention may be chemically synthesized separately and combined using chemical methods to produce the full length molecule.

N-terminal or C-terminal fusion polypeptides or chimeric polypeptides comprising a polypeptide of the invention conjugated with other molecules, (e.g. markers) may be prepared by fusing, through recombinant techniques, the N-terminal or C-terminal of a polypeptide of the invention, and the sequence of a selected molecule with a desired biological function (e.g. marker protein). The resultant fusion proteins contain a polypeptide of the invention fused to the selected molecule as described herein. Examples of molecules which may be used to prepare fusion proteins include immunoglobulins, glutathione-S-transferase (GST), protein A, hemagglutinin (HA), and truncated myc.

Antibodies

Polypeptides of the invention, or cells expressing them can be used as an immunogen to produce antibodies immunospecific for such polypeptides. “Antibodies” as used herein includes monoclonal and polyclonal antibodies, chimeric, single chain, simianized antibodies and humanized antibodies, as well as Fab fragments, including the products of an Fab immunoglobulin expression library.

In an embodiment of the invention, oligopeptides, peptides, or fragments used to induce antibodies to a polypeptide of the invention have an amino acid sequence consisting of at least 5 amino acids and more preferably at least 10 amino acids. The oligopeptides, etc. can be identical to a portion of the amino acid sequence of the natural protein, and they may contain the entire amino acid sequence of a small, naturally occurring molecule. Antibodies having specificity for a polypeptide of the invention may also be raised from fusion proteins created by expressing fusion proteins in bacteria as described herein.

Antibodies including monoclonal and polyclonal antibodies, fragments and chimeras, etc. may be prepared using methods known to those skilled in the art. Antibodies against polypeptides of the invention can be obtained by administering the polypeptides or epitope-bearing fragments, analogues or cells to an animal, preferably a nonhuman, using routine protocols. Monoclonal antibodies may be obtained by any technique known in the art that provides antibodies produced by continuous cell line cultures. (See for example, Kohler, G. and Milstein, C., Nature 256. 495-497 (1975); Kozbor et al.,. Immunology Today 4: 72 (1983); Cole et al., pg. 77-96 in MONOCLONAL ANTIBODIES AND CANCER THERAPY, Alan R. Liss, Inc. (1985).

Single chain antibodies to polypeptides of this invention can be prepared using methods known in the art (e.g. U.S. Pat. No. 4,946,778). Transgenic mice, or other organisms such as other mammals, may be used to express humanized antibodies.

Phage display technology may also be utilized to select antibody genes with binding activities towards a polypeptide of the invention either from repertoires of PCR amplified v-genes of lymphocytes from humans screened for possessing anti-DDHepT or from naive libraries (McCafferty, J. et al., (1990), Nature 348, 552-554; Marks, J. et al., (1992) Biotechnology 10, 779-783). Chain shuffling can also be used to improve the affinity of these antibodies (Clackson, T. et al., (1991) Nature 352, 624-628).

Applications

The polynucleotides, polypeptides, and antibodies of the invention may be used in the prognostic and diagnostic evaluation of disease. (See below). Methods for detecting polynucleotides and polypeptides of the invention, can be used to monitor disease in eukaryotes particularly mammals, and especially humans, particularly those infected or suspected to be infected with an organism comprising a DDhepT gene or polypeptide of the invention, by detecting and localizing the polynucleotides and polypeptides. The applications of the present invention also include methods for the identification of agents (eg. compounds) which modulate the biological activity of a polypeptide of the invention (See below). The compounds, antibodies, etc. may be used for the treatment of disease. (See below).

Diagnostic and Prognostic Methods

A variety of methods can be employed for the diagnostic and prognostic evaluation of disease. Such methods may, for example, utilize polynucleotides of the invention, and fragments thereof, and antibodies of the invention. In particular, the polynucleotides and antibodies may be used, for example, for: (1) the detection of the presence of DDhepT gene mutations, or the detection of either over- or under-expression of DDHepT mRNA relative to a non-disorder state; and (2) the detection of either an over- or an under-abundance of a polypeptide of the invention relative to a non-disorder state or the presence of a modified (e.g., less than full length) polypeptide of the invention.

The methods described herein may be performed by utilizing pre-packaged diagnostic kits comprising at least one specific polynucleotide or antibody described herein, which may be conveniently used, e.g., in clinical settings, to screen and diagnose individuals and to screen and identify or monitor disease in individuals.

Nucleic acid-based detection techniques and peptide detection techniques are described below. The samples that may be analyzed using the methods of the invention include those which are known or suspected to contain a polynucleotide or polypeptide of the invention. The methods may be performed on biological samples including but not limited to cells, lysates of cells which have been incubated in cell culture, genomic DNA (in solutions or bound to a solid support such as for Southern analysis), RNA (in solution or bound to a solid support such as for northern analysis), cDNA (in solution or bound to a solid support), an extract from cells or a tissue (e.g. bone, muscle, cartilage, skin), and biological fluids such as serum, urine, blood, and CSF. The samples may be derived from a patient or a culture.

Methods for Detecting Polynucleotides

The invention provides a process for diagnosing disease, preferably bacterial infections, more preferably infections by Helicobacter pylori, comprising determining from a sample derived from an individual an increased level of expression of a polynucleotide of the invention. Increased or decreased expression of a polynucleotide of the inventon can be measured using any of the methods well known in the art.

A polynucleotide of the invention may be used in southern or northern analysis, dot blot, or other membrane-based technologies; in PCR technologies; or in dipstick, pin, ELISA assays or microarrays utilizing fluids or tissues from patients to detect altered expression. Such qualitative or quantitative methods are well known in the art and some methods are described below.

The polynucleotides of the invention allow those skilled in the art to construct nucleotide probes for use in the detection of polynucleotides of the invention in biological materials. Suitable probes include polynucleotides based on nucleic acid sequences encoding at least 5 sequential amino acids from regions of a polynucleotide of the invention (see SEQ. ID. No. 1, 3, or 5), preferably they comprise 15 to 30 nucleotides. A nucleotide probe may be labeled with a detectable substance such as a radioactive label which provides for an adequate signal and has sufficient half-life such as ³²P, ³H, ¹⁴C or the like. Other detectable substances which may be used include antigens that are recognized by a specific labeled antibody, fluorescent compounds, enzymes, antibodies specific for a labeled antigen, and luminescent compounds. An appropriate label may be selected having regard to the rate of hybridization and binding of the probe to the nucleotide to be detected and the amount of nucleotide available for hybridization. Labeled probes may be hybridized to nucleic acids on solid supports such as nitrocellulose filters or nylon membranes as generally described in Sambrook et al, 1989, Molecular Cloning, A Laboratory Manual (2nd ed.). The nucleic acid probes may be used to detect hepT genes, preferably in human cells. The nucleotide probes may also be useful for example in the diagnosis or prognosis of disease, and in monitoring the progression of a disease condition, or monitoring a therapeutic treatment.

The probe may be used in hybridization techniques to detect DDhepT genes. The technique generally involves contacting and incubating a sample from a patient or other cellular source with a probe of the present invention under conditions favourable for the specific annealing of the probes to complementary sequences in the nucleic acids. After incubation, the non-annealed nucleic acids are removed, and the presence of nucleic acids that have hybridized to the probe if any are detected.

The detection of polynucleotides of the invention may involve the amplification of specific gene sequences using an amplification method such as PCR, followed by the analysis of the amplified molecules using techniques known to those skilled in the art. Suitable primers can be routinely designed by one of skill in the art. (See SEQ ID NO 7 through 14). For example, primers may be designed using commercially available software, such as OLIGO 4.06 Primer Analysis software (National Biosciences, Plymouth Minn.) or another appropriate program, to be about 22 to 30 nucleotides in length, to have a GC content of about 50% or more, and to anneal to the template at temperatures of about 60° C to 72° C.

Genomic DNA may be used in hybridization or amplification assays of biological samples to detect abnormalities in cells involving DDHepT structure, including point mutations, insertions, and deletions. For example, direct sequencing, single stranded conformational polymorphism analyses, heteroduplex analysis, denaturing gradient gel electrophoresis, chemical mismatch cleavage, and oligonucleotide hybridization may be utilized. Mutations in the DNA sequence of a DDhepT gene may be used to diagnose infection and to serotype and/or classify the infectious agent

Genotyping techniques known to one skilled in the art can be used to type polymorphisms that are in close proximity to the mutations in a DDhepT gene. The polymorphisms may be used to identify species of organisms that are likely to cause disease.

RT-PCR may be used to detect mutations in the RNA. In particular, RT-PCR may be used in conjunction with automated detection systems such as for example GeneScan.

The primers and probes may be used in the above described methods in situ i.e directly on tissue sections (fixed and/or frozen) of patient tissue obtained from biopsies or resections.

Oligonucleotides derived from any of the polynucleotides of the invention may be used as targets in microarrays. “Microarray” refers to an array of distinct polynucleotides or oligonucleotides synthesized on a substrate, such as paper, nylon, or other type of membrane, filter, chip, glass slide, or any other suitable solid support.

The microarrays can be used to monitor the expression level of large numbers of genes simultaneously (to produce a transcript image) and to identify genetic variants, mutations, and polymorphisms. This information can be useful in determining gene function, diagnosing disease, and in developing and monitoring the activity of therapeutic agents (Heller, R. et al. (1997) Proc. Natl. Acad, Sci. 94:2150-55).

The polynucleotides of the present invention are useful for chromosome identification. The sequences can be specifically targeted to, and can hybridize with a particular location on an individual microbial chromosome, particularly a Helicobacter pylori chromosome. The mapping of relevant sequences to a chromosome is an important step in correlating those sequences with genes associated with microbial pathogenicity and disease, or to precise chromosomal regions critical to the growth, survival, and/or ecological niche of an organism. The physical position of the sequence on the chromosome can be correlated with genetic map data to define a genetic relationship between the gene and another gene or phenotype by, for example, linkage analysis.

Differences in the RNA or genomic sequence between microbes of different phenotypes may also be determined. A mutation or sequence observed in some or all of the organisms of a certain phenotype but not in organisms lacking that phenotype, will likely be the causative agent for the phenotype. Thus, chromosomal regions may be identified that confer pathogenicity, growth characteristics, survival characteristics, and/or ecological niche.

The polynucleotides of the invention may be used in differential screening and differential display methods known in the art. (e.g. see Chuang et al J. Bacteriol. 175: 2026, 1993). Genes are identified which are expressed in an organism by identifying mRNA present using randomly primed RT-PCR. Pre-infection and post-infection profiles are compared to identify genes up and down regulated during infection.

Methods for Detecting Polypeptides

Antibodies specifically reactive with a polypeptide of the invention or derivatives thereof, such as enzyme conjugates or labeled derivatives, may be used to detect the polypeptides in various samples. They may be used as diagnostic or prognostic reagents and they may be used to detect abnormalities in the level of a polypeptide of the invention, or abnormalities in the structure of the polypeptides. Antibodies may also be used to screen potentially therapeutic compounds in vitro to determine their effects on a disease. In vitro immunoassays may also be used to assess or monitor the efficacy of particular therapies. The antibodies of the invention may also be used in vitro to determine the level of DDHepT expression in cells genetically engineered to produce a DDHepT.

In an embodiment, the invention provides a diagnostic method for detecting over-expression of a polypeptide of the invention compared to normal control tissue samples. The method may be used to detect the presence of an infection.

The antibodies may be used in any known immunoassays which rely on the binding interaction between an antigenic determinant of a polypeptide of the invention, and the antibodies. Examples of such assays are radioimmunoassays, enzyme immunoassays (e.g. ELISA), immunofluorescence, immunoprecipitation, latex agglutination, hemagglutination, and histochemical tests. The antibodies may be used to detect and quantify polypeptides of the invention in a sample in order to determine its role in particular cellular events or pathological states, and to diagnose and treat such pathological states.

Antigenic polypeptides of the invention or fragments thereof may be used in immunoassays to detect antibody levels and correlations can be made with diseases such as gastroduodenal disease and with duodenal ulcer in particular. Immunoassays based on well defined recombinant antigens can be developed. Antibodies to Helicobacter pylori HepT polypeptides within biological samples such as blood or serum samples may be detected.

The antibodies of the invention may be used in immuno-histochemical analyses, for example, at the cellular and sub-subcellular level, to detect a polypeptide of the invention, to localise it to particular cells and tissues, and to specific subcellular locations, and to quantitate the level of expression.

Cytochemical techniques known in the art for localizing antigens using light and electron microscopy may be used to detect a polypeptide of the invention. Generally, an antibody of the invention may be labeled and a polypeptide may be localised in tissues and cells based upon detection of the label.

Various methods of labeling polypeptides are known in the art and may be used to label antibodies and polypeptides of the invention. Examples of detectable substances include, but are not limited to, the following: radioisotopes (e.g., ³H, ¹⁴C, ³⁵S, ¹²⁵, ¹³¹I), fluorescent labels (e.g., PITC, rhodamine, lanthanide phosphors), luminescent labels such as luminol; enzymatic labels (e.g., horseradish peroxidase, β-galactosidase, luciferase, alkaline phosphatase, acetylcholinesterase), biotinyl groups (which can be detected by marked avidin e.g., streptavidin containing a fluorescent marker or enzymatic activity that can be detected by optical or calorimetric methods), and predetermined polypeptide epitopes recognized by a secondary reporter (e.g., leucine zipper pair sequences, binding sites for secondary antibodies, metal binding domains, epitope tags). In some embodiments, labels are attached via spacer arms of various lengths to reduce potential steric hindrance. Antibodies may also be coupled to electron dense substances, such as ferritin or colloidal gold, which are readily visualised by electron microscopy.

An antibody or sample may be immobilized on a carrier or solid support which is capable of immobilizing cells, antibodies etc. For example, the carrier or support may be nitrocellulose, or glass, polyacrylamides, gabbros, and magnetite. The support material may have any possible configuration including spherical (e.g. bead), cylindrical (e.g. inside surface of a test tube or well, or the external surface of a rod), or flat (e.g. sheet, test strip). Indirect methods may also be employed in which the primary antigen-antibody reaction is amplified by the introduction of a second antibody, having specificity for the antibody reactive against a polypeptide of the invention. By way of example, if the antibody having specificity against a polypeptide of the invention is a rabbit IgG antibody, the second antibody may be goat anti-rabbit gamma-globulin labelled with a detectable substance as described herein.

Where a radioactive label is used as a detectable substance, a polypeptide of the invention may be localized by radioautography. The results of radioautography may be quantitated by determining the density of particles in the radioautographs by various optical methods, or by counting the grains.

A polypeptide of the invention may also be detected by assaying for DDHepT activity as described herein. For example, a sample may be reacted with an acceptor molecule and a donor molecule under conditions where a DDHepT is capable of transferring the donor molecule to the acceptor molecule to produce a donor-acceptor complex.

Methods for Identifying or Evaluating Substances/Compounds

The invention provides methods for identifying substances that modulate the biological activity of a polypeptide of the invention including substances that interfere with, or enhance the activity of the polypeptide.

The substances and compounds identified using the methods of the invention include but are not limited to peptides such as soluble peptides including Ig-tailed fusion peptides, members of random peptide libraries and combinatorial chemistry-derived molecular libraries including libraries made of D- and/or L-configuration amino acids, phosphopeptides (including members of random or partially degenerate, directed phosphopeptide libraries), antibodies [e.g. polyclonal, monoclonal, humanized, antisense, oligosaccharides, anti-idiotypic, chimeric, single chain antibodies, fragments, (e.g. Fab, F(ab)₂, and Fab expression library fragments, and epitope-binding fragments thereof)], and small organic or inorganic molecules. The substance or compound may be an endogenous physiological compound or it may be a natural or synthetic compound. A substance of the invention may be a natural substrate or ligand (e.g. an acceptor or donor molecule) or a structural or functional mimetic. The substance may be a small molecule ligand in, for example, cells, cell-free preparations, chemical libraries, and natural product mixtures

Substances which modulate a polypeptide of the invention can be identified based on their ability to associate with (or bind to) a polypeptide of the invention. Therefore, the invention also provides methods for identifying substances which associate with a polypeptide of the invention. Substances identified using the methods of the invention may be isolated, cloned and sequenced using conventional techniques. A substance that associates with a polypeptide of the invention may be an agonist or antagonist of the biological or immunological activity of the polypeptide.

The term “agonist”, refers to a molecule that increases the amount of, or prolongs the duration of, or the activity of the polypeptide. The term “antagonist” refers to a molecule which decreases the biological or immunological activity of the polypeptide. Agonists and antagonists may include proteins, nucleic acids, carbohydrates, or any other molecules that associate with a polypeptide of the invention (including ligands or mimetics thereof).

Substances which can associate with a polypeptide of the invention may be identified by reacting the polypeptide with a test substance which potentially associates with the polypeptide, under conditions which permit the association, and removing and/or detecting polypeptide associated with the test substance. Substance-polypeptide complexes, free substance, or non-complexed polypeptide may be assayed, or the activity of the polypeptide may be assayed. Conditions which permit the formation of substance-polypeptide complexes may be selected having regard to factors such as the nature and amounts of the substance and the polypeptide.

The substance-polypeptide complex, free substance or non-complexed polypeptide may be isolated by conventional isolation techniques, for example, salting out, chromatography, electrophoresis, gel filtration, fractionation, absorption, polyacrylamide gel electrophoresis, agglutination, or combinations thereof. To facilitate the assay of the components, antibody against a polypeptide of the invention or the substance, or labeled polypeptide, or a labeled substance may be utilized. The antibodies, polypeptide, or substances may be labeled with a detectable substance as described above.

A polypeptide of the invention, or the substance used in the method of the invention may be insolubilized. For example, a polypeptide, or substance may be bound to a suitable carrier such as agarose, cellulose, dextran, Sephadex, Sepharose, carboxymethyl cellulose polystyrene, filter paper, ion-exchange resin, plastic film, plastic tube, glass beads, polyamine-methyl vinyl-ether-maleic acid copolymer, amino acid copolymer, ethylene-maleic acid copolymer, nylon, silk, etc. The carrier may be in the shape of, for example, a tube, test plate, beads, disc, sphere etc. The insolubilized polypeptide or substance may be prepared by reacting the material with a suitable insoluble carrier using known chemical or physical methods, for example, cyanogen bromide coupling.

The invention also contemplates a method for evaluating a compound for its ability to modulate the biological activity of a polypeptide of the invention, by assaying for an agonist or antagonist (i.e. enhancer or inhibitor) of the association of the polypeptide with a substance which associates with the polypeptide. The basic method for evaluating if a compound is an agonist or antagonist of the association of a polypeptide of the invention and a substance that associates with the polypeptide, is to prepare a reaction mixture containing the polypeptide and the substance under conditions which permit the formation of substance-polypeptide complexes, in the presence of a test compound. The test compound may be initially added to the mixture, or may be added subsequent to the addition of the polypeptide and substance. Control reaction mixtures without the test compound or with a placebo are also prepared. The formation of complexes is detected and the formation of complexes in the control reaction but not in the reaction mixture indicates that the test compound interferes with the interaction of the polypeptide and substance. The reactions may be carried out in the liquid phase or the polypeptide, substance, or test compound may be immobilized as described herein. In an embodiment of the invention, the substance is a natural substrate or ligand of a polypeptide of the invention, or a structural or functional mimetic thereof.

It will be understood that the agonists and antagonists i.e. inhibitors and enhancers that can be assayed using the methods of the invention may act on one or more of the binding sites on the polypeptide or substance including agonist binding sites, competitive antagonist binding sites, non-competitive antagonist binding sites or allosteric sites.

The invention also makes it possible to screen for antagonists that inhibit the effects of an agonist of the interaction of a polypeptide of the invention with a substance which is capable of associating with or binding to the polypeptide. Thus, the invention may be used to assay for a compound that competes for the same binding site of a polypeptide of the invention.

In an embodiment, the invention provides a method of screening compounds to identify those which enhance (agonist) or block (antagonist) the action of polypeptides or polynucleotides of the invention, particularly those compounds that are bacteriostatic and/or bacteriocidal. The method of screening may involve high-throughput techniques. For example, to screen for agonists or antagoists, a synthetic reaction mix, a cellular compartment, such as a membrane, cell envelope or cell wall, or a preparation of any thereof, comprising a polypeptide of the invention and a labeled substrate or ligand of such polypeptide is incubated in the absence or the presence of a test compound that may be an agonist or antagonist. The ability of the test compound to agonize or antagonize the polypeptide is reflected in decreased binding of the labeled ligand or decreased production of product from such substrate. Molecules that bind gratuitously, i.e., without inducing the effects of a polypeptide of the invention are most likely to be good antagonists. Molecules that bind well and increase the rate of product production from substrate are agonists. Detection of the rate or level of production of product from substrate may be enhanced by using a reporter system. Reporter systems that may be useful in this regard include but are not limited to calorimetric labeled substrate converted into product, a reporter gene that is responsive to changes in polynucleotide or polypeptide activity, and binding assays known in the art.

Another example of an assay for antagonists is a competitive assay that combines a polypeptide of the invention and a potential antagonist with molecules that bind a polypeptide of the invention, a recombinant binding molecule, natural substrate or ligand, or substrate or ligand mimetic, under appropriate conditions for a competitive inhibition assay. The polypeptide can be labeled, such as by radioactivity or a colorimetric compound, such that the number of polypeptides bound to a binding molecule or converted to product can be determined accurately to assess the effectiveness of the potential antagonist.

Agents that modulate a polypeptide of the invention can be identified based on their ability to interfere with or enhance the activity of a polypeptide of the invention. Therefore, the invention provides a method for evaluating a compound for its ability to modulate the activity of a polypeptide of the invention comprising (a) reacting an acceptor molecule and a donor molecule for a polypeptide of the invention in the presence of a test compound; (b) measuring transfer of a sugar of the donor molecule to the acceptor molecule, and (c) carrying out steps (a) and (b) in the absence of the test compound to determine if the compound interferes with or enhances transfer of the sugar of the donor molecule to the acceptor molecule by the polypeptide.

The acceptor molecule or donor molecule may be labeled with a detectable substance as described herein, and the interaction of the polypeptide of the invention with the acceptor molecule and donor molecule will give rise to a detectable change. The detectable change may be colorimetric, photometric, radiometric, potentiometric, etc. The activity of a polypeptide of the invention may also be determined using methods based on HPLC (Koenderman et al., FEBS Lett. 222:42, 1987) or methods employing synthetic oligosaccharide acceptors attached to hydrophobic aglycones (Palcic et al Glycoconjugate 5:49, 1988; and Pierce et al, Biochem. Biophys. Res. Comm. 146: 679,1987).

A polypeptide of the invention is reacted with the acceptor and donor molecules at a pH and temperature and in the presence of a metal cofactor, usually a divalent cation, effective for the polypeptide to transfer the sugar of the donor molecule to the acceptor molecule, and where one of the components is labeled, to produce a detectable change. It is preferred to use a buffer with the acceptor and donor molecules to maintain the pH within the pH range effective for the proteins. The buffer, acceptor and donor molecules may be used as an assay composition. Other compounds such as EDTA and detergents may be added to the assay composition. The polypeptide may be obtained from natural sources or produced using recombinant methods as described herein.

The reagents suitable for applying the methods of the invention to evaluate compounds that modulate a polypeptide of the invention may be packaged into convenient kits providing the necessary materials packaged into suitable containers. The kits may also include suitable supports useful in performing the methods of the invention.

A substance that inhibits a polypeptide may be identified by treating a cell which expresses the polypeptide with a test substance, and analyzing the lipopolysaccharide structures on the cell. Lipopolysaccharide can be analyzed using the methods described herein. Cells that have not been treated with the substance or which do not express the polypeptide may be employed as controls.

Substances which inhibit transcription or translation of a DDhepT gene may be identified by transfecting a cell with an expression vector comprising a recombinant molecule of the invention, including a reporter gene, in the presence of a test substance and comparing the level of expression of a DDhepT, or the expression of the protein encoded by the reporter gene with a control cell transfected with the nucleic acid molecule in the absence of the substance. The method can be used to identify transcription and translation inhibitors of a DDhepT gene.

Compositions and Treatments

The polynucleotides and polypeptides of the invention and substances or compounds identified by the methods described herein, antibodies, and antisense nucleic acid molecules of the invention may be used to treat diseases. Examples of diseases that may be treated include diseases associated with organisms that contain a polypeptide or polynucleotide of the present invention. In an embodiment the organsims are from the Helicobacter family, and are particularly Helicobacter pylori species.

Helicobacter pylori infects the stomachs of over one-third of the world's population causing stomach cancer, ulcers, and gastritis (International Agency for Research on Cancer (1994) Schistosomes, Liver Flukes and Helicobacter Pylori (International Agency for Research on Cancer, Lyon, France; http://www.uicc.ch/ecp/ecp2904.htm). There is also a recognized cause-and-effect relationship between H. pylori and gastric adenocarcinoma, classifying the bacterium as a Group I (definite) carcinogen. Preferred agonists of the invention found using screens provided by the invention, particularly broad-spectrum antibiotics, will be useful in the treatment of H. pylori infection, and they should decrease the advent of H. pylori-induced cancers, such as gastrointestinal carcinoma. The agonists should also be useful in the treatment of gastric ulcers and gastritis.

Accordingly, the proteins, substances, antibodies, and compounds etc. may be formulated into pharmaceutical compositions for administration to subjects in a biologically compatible form suitable for administration in vivo. By “biologically compatible form suitable for administration in vivo” is meant a form of the substance to be administered in which any toxic effects are outweighed by the therapeutic effects. The substances may be administered to living organisms including humans, and animals. Administration of a therapeutically active amount of the pharmaceutical compositions of the present invention is defined as an amount effective, at dosages and for periods of time necessary to achieve the desired result. For example, a therapeutically active amount of a substance may vary according to factors such as the disease state, age, sex, and weight of the individual, and the ability of antibody to elicit a desired response in the individual. Dosage regima may be adjusted to provide the optimum therapeutic response. For example, several divided doses may be administered daily or the dose may be proportionally reduced as indicated by the exigencies of the therapeutic situation.

The active substance may be administered in a convenient manner such as by injection (subcutaneous, intravenous, etc.), oral administration, inhalation, transdermal application, or rectal administration. Depending on the route of administration, the active substance may be coated in a material to protect the compound from the action of enzymes, acids and other natural conditions that may inactivate the compound.

The compositions described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the compositions include, albeit not exclusively, solutions of the substances or compounds in association with one or more pharmaceutically acceptable vehicles or diluents, and contained in buffered solutions with a suitable pH and iso-osmotic with the physiological fluids.

After pharmaceutical compositions have been prepared, they can be placed in an appropriate container and labeled for treatment of an indicated condition. For administration of a composition of the invention the labeling would include amount, frequency, and method of administration.

The compositions, substances, compounds etc. may be indicated as therapeutic agents either alone or in conjunction with other therapeutic agents or other forms of treatment (e.g. chemotherapy or radiotherapy). By way of example, they can be used in combination with anti-proliferative agents, antimicrobial agents, immunostimulatory agents, or anti-inflammatories. In particular, they can be used in combination with anti-bacterial agents. They can be administered concurrently, separately, or sequentially with other therapeutic agents or therapies.

Polynucleotides of the invention or any fragment thereof or antisense sequences may be used for therapeutic purposes. Antisense to a polynucleotide encoding a polypeptide of the invention may be used in situations to block the synthesis of the polypeptide. In particular, cells may be transformed with sequences complementary to polynucleotides of the invention. Thus, antisense sequences may be used to modulate activity of a polypeptide of the invention, or to achieve regulation of gene function. Sense or antisense oligomers or larger fragments, can be designed from various locations along the coding or regulatory regions of sequences encoding a polypeptide of the invention.

Expression vectors may be derived from retroviruses, adenoviruses, herpes or vaccinia viruses or from various bacterial plasmids for delivery of nucleic acid sequences to the target organ, tissue, or cells. Vectors that express antisense nucleic acid sequences of DDhepT can be constructed using techniques well known to those skilled in the art (see for example, Sambrook et al. (supra)).

Genes encoding a DDHepT can be turned off by transforming a cell or tissue with expression vectors that express high levels of a polynucleotide of the invention. Such constructs may be used to introduce untranslatable sense or antisense sequences into a cell. Even if they do not integrate into the DNA, the vectors may continue to transcribe RNA molecules until all copies are disabled by endogenous nucleases.

Modification of gene expression may be achieved by designing antisense molecules, DNA, RNA, or Peptide nucleic acid (PNA), to the control regions of a DDHepT gene i.e. the promoters, enhancers, and introns. Preferably the antisense molecules are oligonucleotides derived from the transcription initiation site (e.g. between positions −10 and +10 from the start site). Inhibition can also be achieved by using triple-helix base-pairing techniques. Triple helix pairing causes inhibition of the ability of the double helix to open sufficiently for the binding of polymerases, transcription factors, or regulatory molecules (see Gee J. E. et al (1994) In: Huber, B. E. and B. I. Carr, Molecular and Immunologic Approaches, Futura Publishing Co., Mt. Kisco, N.Y.). An antisense molecule may also be designed to block translation of mRNA by inhibiting binding of the transcript to the ribosomes.

Ribozymes may be used to catalyze the specific cleavage of RNA. Ribozyme action involves sequence-specific hybridization of the ribozyme molecule to complementary target RNA, followed by endonucleolytic cleavage. For example, hammerhead motif ribozyme molecules may be engineered that can specifically and efficiently catalyze endonucleolytic cleavage of sequences encoding a polypeptide of the invention.

Specific ribosome cleavage sites within any RNA target may be initially identified by scanning the target molecule for ribozyme cleavage sites which include the following sequences: GUA, GUU, and GUC. Short RNA sequences of between 15 and 20 ribonucleotides corresponding to the region of the cleavage site of the target gene may be evaluated for secondary structural features which may render the oligonucleotide inoperable. The suitability of candidate targets may be evaluated by testing accessibility to hybridization with complementary oligonucleotides using ribonuclease protection assays.

Therapeutic efficacy and toxicity may be determined by standard pharmaceutical procedures in cell cultures or with experimental animals, such as by calculating the ED₅₀ (the dose therapeutically effective in 50% of the population) or LD₅₀ (the dose lethal to 50% of the population) statistics. The therapeutic index is the dose ratio of therapeutic to toxic effects and it can be expressed as the ED₅/LD₅₀ ratio. Pharmaceutical compositions which exhibit large therapeutic indices are preferred.

Mutant Organisms

The invention provides novel mutants of Helicobacter bacteria, in particular mutants of H. pylori, having mutated (deactivated) DDhepT genes. In general, “mutated” refers to a sudden heritable change in the phenotype of an organism which can be spontaneous or induced by known mutagenic agents, including radiation and various chemicals.

Methods are known in the art that can be used to generate mutations to produce the mutant bacteria of the present invention. For example, the transposon, Tn10, can be used to produce chromosomal deletions in a wide variety of bacteria (Kleckner et al., J. Mol. Biol. 116:125-159, 1977; EPO Pub. No. 315,682; U.S. Pat. No. 5,387,744. Alternatively, methods may be used that involve introducing specific deletions in a DDhepT gene in an organism. A specific deletion in the selected gene can be generated by either of two general methods.

The first method generates a mutation in a gene isolated from a population of clones contained in a genomic DNA library using restriction enzymes and the second method generates the mutation in a gene of known sequence using PCR. Using the first method, the position of the gene on a vector is identified using transposon tagging and a restriction map of the recombinant DNA in the vector is generated. Information derived from the transposon tagging allows all or a portion of a gene to be excised from the vector using the known restriction enzyme sites.

The second method is based upon PCR. Divergent PCR primers are used to amplify the upstream and downstream regions flanking a specified segment of the DDhepT DNA to be deleted from the gene, generating a PCR product consisting of the cloning vector and upstream and downstream flanking nucleotide sequences (Innes et al. Eds., PCR Protocols, 1990, Academic Press, New York). In a variation of this method, PCR products are produced representing portions of the gene or flanking sequence, which are then joined together in a cloning vector.

Mutagenesis of a cloned DDhepT gene may also be carried out by insertion of a marker into an insertion site in the gene. For example, a kanamycin resistance marker may be ligated into an insertion site created in a DDhepT gene by reverse PCR (See Example 1).

The DNA containing the mutant gene can be introduced into the bacterial host by transformation using chemical means or electroporation, by recombinant phage infection, or by conjugation. In preferred embodiments the mutant gene is introduced into the chromosomes of the bacteria which can be accomplished using any of a number of methods well known in the art such as, for example, methods using temperature-sensitive replicons (Hamilton et al., J. Bacteriol. 171:4617-4622, 1989), linear transformation of recBC mutants (Jasin et al., J. Bacteriol. 159:783-786, 1984), or host restricted replicons known as suicide vectors (Miller et al., J. Bacteriol. 170:2575-2583, 1988). The particular method used is coupled with an appropriate counter selection method such as, for example, by using PCR, nucleic acid hybridization, or an immunological method.

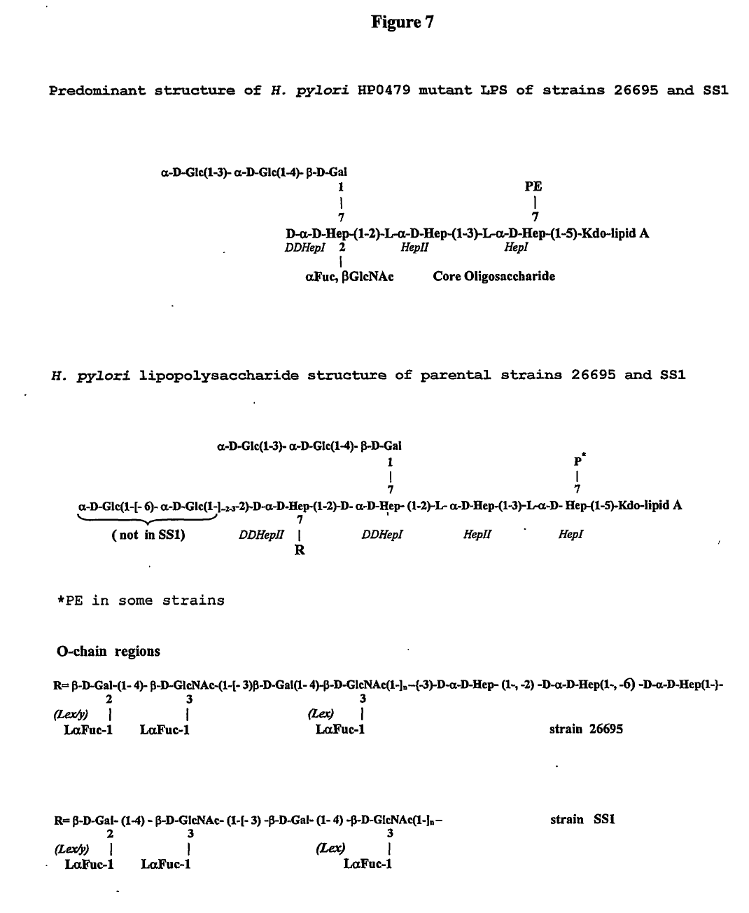

Mutant bacteria of the invention include H. plyori 0479GM1; H. pylori 0479M1, H. pylori 04793M1, H. pylori 0479TM1, and H. pylori 0479PM1. Structural analysis of LPS isolated from the mutants confirmed that O-chain synthesis has been affected by the mutations and revealed the exact structure of the truncated LPS molecules (see FIG. 7). The mutant strains were also shown to have a reduced capacity of gastric colonization.

The invention also provides modified LPS molecules from mutants of the invention. The modified LPS may be isolated from the mutant bacteria and at least partially purified using techniques well known to those skilled in the art. Preparations of at least 70%, particularly 80%, more particularly 90%, most particularly 95% pure LPS are preferred. The purity of an LPS preparation is expressed as the weight percentage of the total Helicobacter antigens present in the preparation. The purified LPS can be used as antigen either directly or after being conjugated to a suitable carrier protein. The structures of LPS of mutant bacteria are shown in FIG. 7.

Methods for Preparing Oligosaccharides

The invention relates to a method for preparing an oligosaccharide comprising contacting a reaction mixture comprising an activated donor molecule and an acceptor molecule in the presence of a polypeptide of the invention.

In an embodiment of the invention, the oligosaccharides are prepared on a carrier that is non-toxic to a mammal, in particular a lipid isoprenoid or polyisoprenoid alcohol. An example of a suitable carrier is dolichol phosphate. The oligosaccharide may be attached to a carrier via a labile bond allowing for chemical removal of the oligosaccharide from the lipid carrier. In the alternative, the oligosaccharide transferase may be used to transfer the oligosaccharide from a lipid carrier to a protein.

Vaccines

The mutant bacteria expressing the truncated LPS and the modified LPS isolated from such mutants are useful sources of antigens in vaccination against Helicobacter bacteria, in particular against H. pylori. Such vaccines are normally prepared from dead bacterial cells, using methods well known to those skilled in the art, and usually contain various auxiliary components, such as an appropriate adjuvant and a delivery system. A delivery system aiming at mucosal delivery is preferred. Preferably but not essentially, the antigenic preparation is administered orally to the host, but parenteral administration is also possible. Live vaccines based on H. pylori mutants may also be prepared, but would normally require an appropriate vector for mucosal delivery. Vaccines of the present invention are useful in preventing and reducing the number of H. pylori infections and indirectly in reducing the incidence of pathological conditions associated with such infections, in particular gastric cancer.

Another aspect of the invention relates to a method for inducing an immunological response in an individual, particularly a mammal which comprises inoculating the individual with an antigen (e.g. modified LPS) adequate to produce antibody and/ or T cell immune response to protect said individual from infection, particularly bacterial infection and most particularly Helicobacter pylori infection. Also provided are methods whereby such immunological response slows bacterial replication.

A further aspect of the invention relates to an immunological composition which, when introduced into an individual capable of having induced within it an immunological response, induces an immunological response in such individual to Helicobacter wherein the composition comprises a modified LPS. The immunological response may be used therapeutically or prophylactically and may take the form of antibody immunity or cellular immunity such as that arising from CTL or CD4+T cells.

A modified LPS may be fused with a molecule which may not by itself produce antibodies, but is capable of stabilizing the modified LPS and producing an antigen which will have immunogenic and protective properties. Examples of such molecules are lipoprotein D from Hemophilus influenzae, glutathione-S-transferase (GST) or beta-galactosidase. Moreover, the molecule may act as an adjuvant in the sense of providing a generalized stimulation of the immune system.

The invention provides methods using the modified LPS in immunization experiments in animal models of infection with Helicobacter to identify epitopes able to provoke a prophylactic or therapeutic immune response. It is believed that this approach will allow for the subsequent preparation of monoclonal antibodies of particular value from the requisite organ of the animal successfully resisting or clearing infection for the development of prophylactic agents or therapeutic treatments of bacterial infection, particularly Helicobacter pylori infection, in mammals, particularly humans.

The modified LPS may be used as an antigen for vaccination of a host to produce specific antibodies which protect against invasion of bacteria, for example by preventing colonization.

The invention also includes a vaccine formulation which comprises a modified LPS of the invention together with a suitable carrier. The formulation is preferably administered parenterally, including, for example, administration that is subcutaneous, intramuscular, intravenous, or intradermal. Formulations suitable for parenteral administration include aqueous and non-aqueous sterile injection solutions which may contain anti-oxidants, buffers, bacteriostats and solutes which render the formulation insotonic with the bodily fluid, preferably the blood, of the individual; and aqueous and non-aqueous sterile suspensions which may include suspending agents or thickening agents. The formulations may be presented in unit-dose or multi-dose containers, for example, sealed ampules and vials and may be stored in a freeze-dried condition requiring only the addition of the sterile liquid carrier immediately prior to use. The vaccine formulation may also include adjuvant systems for enhancing the immunogenicity of the formulation, such as oil-in water systems and other systems known in the art. The dosage will depend on the specific activity of the vaccine and can be readily determined by routine experimentation.

The following non-limiting example is illustrative of the present invention:

### EXAMPLE 1

Materials and Methods

Bacterial Strains and Media H. pylori strains used are listed in Table 1. H. pylori cultures were grown either on solid Columbia Blood agar (Difco) supplemented with Horse Blood (5%), Vancomycin (10 mg/L), Nalidixic acid (1.1 mg/L;), Bacitracin (20 mg/L), Polymyxin B (0.33 mg/L), and Amphotericin A (5 mg/L) or in Brucella broth supplemented with Fetal Bovine Calf Serum (5-10%), Vancomycin (10 mg/L), Nalidixic acid (1.1 mg/L;), Bacitracin (20 mg/L), Polymyxin B (0.33 mg/L), and Amphotericin A (5 mg/L). All H. pylori cultures were incubated at 37° C. in a tri-gas incubator with a gas mixture of 85% N₂, 10% CO₂ and 5% O₂ until the desired amount of growth was obtained, normally 2-3 days. For liquid media cultures were also shaken at 100 rpm.

For propagation and maintenance of plasmids, E. coli DH5α was used (Table 1). E. coli cultures were grown in Luria broth supplemented, when needed, with Ampicillin (100 mg/L; SIGMA), X-gal (20 mg/L; Gibco/BRL), IPTG (100 mM; Gibco/BRL), or Kanamycin (20 mg/L; SIGMA).

Plasmids The plasmid pUC19 was used to clone PCR-amplified H. pylori genes as well as a suicide vector to transform H. pylori strains. For mutagenesis, a 1398 bp SmaI fragment from the plasmid pIP1433 (Labigne-Roussel, et al., 1988) was used which contained a Campylobacter coli Kanamycin marker.

Polymerase Chain Reaction (PCR) Amplification of H. pylori HP0479 Amplification of the H. pylori HP0479 gene for cloning was carried out using the heat stable DNA polymerase PwoI (Roche Biochemicals). This enzyme also carries a 3′-5′ proofreading activity which increases the fidelity of replication and produces a blunt-ended product. The primers used for this purpose, BP0479-F1 (SEQ ID NO: 7) and HP0479-R1 (SEQ ID NO: 8), are listed in Table 2. An annealing temperature of 60° C. was used for amplification. PCR amplification was carried out on the cloned HP0479 H. pylori genes to generate blunt ends for insertion of a C. jejuni Kanamycin cassette for mutagenesis. PwoI was used for this purpose. The primers HP0479-mutF1 (SEQ ID NO: 9) and HP0479-mutR1 (SEQ ID NO: 10) were used for amplification (Table 2). PCR using Taq DNA polymerase (Roche Molecular Biochemicals) was used to ascertain if the C. jejuni Kanamycin marker had been inserted into the appropriate genes. The primers [HP0479-GF1 (SEQ ID NO: 11) and HP0479-GR1 (SEQ ID NO: 12)] and annealing temperatures used for amplification are listed in Table 2.

Cloning of the HP0479 gene The HP0479 gene of H. pylori 26695 was cloned using the cloning vector pUC19. Polymerase chain reaction (PCR) was used to amplify the gene from genomic DNA of H. pylori 26695, as well as the HP0479 homologs from the genomes of the type strain (ATCC 43504), 0:3, PJ1 and Sydney strain. The amplified PCR fragments were analyzed by agarose gel electrophoresis to insure that a product of the expected size was obtained. Subsequently, the PCR fragments were cloned into the SmaI site of the pUC19 vector using T4 DNA ligase with protocols described by the manufacturer (Gibco/BRL). The ligation mixture was used to transform E. coli DH5α using methods described by Chung and Miller (1988). Selection of clones was carried out using standard white/blue selection on LB agar supplemented with X-gal, IPTG and Ampicillin. Plasmid DNA was isolated and purified using a plasmid isolation kit (Qiagen). Insertion of the gene was confirmed and orientation of insertion was determined by restriction endonuclease analysis using the enzyme HindIII. One clone, p0479-1, carrying the HP0479 gene from H. pylori 26695, was used for mutagenesis. The cloned HP0479 gene and the cloned HP0479 homologs from the other strains were sequenced for comparisons at the DNA and amino acid levels.

Mutagenesis of the cloned HP0479 gene Mutagenesis of the cloned HP0479 gene was carried out by the insertion of a Kanamycin resistance marker from Campylobacter coli, carried on a 1348 bp SmaI fragment from the plasmid pIP1433 (Labigne-Roussel, et al., 1988). A blunt-ended insertion site was created in the cloned HP0479 gene by reverse PCR using primers HP0479-mutF1 and HP0479-mutR1. The Kanamycin cassette was ligated into the insertion site using T4 ligase. The ligation mix was used to transform DH5α, as above (Chung and Miller, 1988), and selection was carried out on LB-agar supplemented with Ampicillin and Kanamycin. Insertion of the Kanamycin cassette and orientation of insertion was confirmed by restriction endonuclease analysis using the enzyme EcoRI. One plasmid, p0479-K1, was used for transformation of H. pylori stains.

Transformation of H. pylori with p0479-K1 Natural transformation of H. pylori was carried out following a protocol modified from those of Haas et al., 1993. Briefly, H. pylori cultures were grown on Columbia Blood agar as described above, for 2-3 days. Bacterial growth was suspended in 3 ml of Brucella broth (see media above) and adjusted to an optical density (OD₆₀₀) of 0.1-0.2 with Brucella broth. One ml of diluted culture was mixed with 10-50 μl (5-25 μg) of plasmid DNA in a sterile 24-well cell-culture dish. The cells were incubated at 37° C. for 4-6 hours in the tri-gas incubator. Aliquots of the transformed cells were then plated onto Columbia Blood agar with Kanamycin and the plates were incubated in the tri-gas incubator for 3-7 days until colonies were visible. Individual colonies were picked with a sterile loop and streaked onto fresh Columbia Blood agar (+Kanamycin) plates and incubated at 37° C. for 2-3 days in the tri-gas incubator. The streaked cultures were suspended in 300 μl of Brucella broth and plated onto fresh plates. The plates were incubated at 37° C. for 2-3 days in the tri-gas incubator. The cultures were then suspended in 3 ml of Brucella broth and two 300 μl aliquots were taken for crude LPS analysis and DNA isolation. Glycerol was added to the remaining suspension to a final concentration of 15%. The cells were divided into 200 μL aliquots, flash frozen and stored at −82° C.

Mutagenesis of HP0480 Polarity effects were analyzed by mutation of the gene immediately downstream of HP0479. This gene was amplified from the H. pylori 26695 genome using the primers HP0480-F1 (GATAACCTCATCACGCTTAG) (SEQ ID NO: 13) and HP0480-R1 (TTCAATCCATTCTAACGC) (SEQ ID NO: 14) with PwoI as described above with an annealing temperature of 60° C. The gene was cloned into pUC19 and mutated by insertion of the C. coli Chloramphenicol cassette from the plasmid pRY109 (Yao et al., 1993) into a unique SmaI restriction site. The mutated gene was transformed into 26695 and Sydney strain as described above and the LPS of whole cells was analyzed by SDS-PAGE.

SDS-PAGE Electrophoresis and Western Blotting LPS samples were prepared from whole cells following a method described by Logan and Trust, 1984. Samples were electrophoresed according to the methods of Laemmli(1970) on 12% SDS-PAGE gels using a mini-slab gel apparatus (BioRad). LPS was visualized using the silver staining technique described by Tsai and Frasch (1982). Western blots of LPS gels were carried out using the protocol of Logan and Trust (1984) with anti-Lewis monoclonal antibodies (Signet Laboratories). The antibodies were used at a dilution of 1:500.

Membrane fraction analysis Membrane fractions were prepared from overnight (18 h) liquid cultures of HP0479 mutant strains and parental strains using the protocols described by Logan et al. (2000). The fractions were analyzed by SDS-PAGE and stained using Coomasie blue.

Preparation of LPS H. pylori strains were cultivated as previously described (Logan et al. 2000). The wet cell mass obtained by centrifugation of the bacterial growth was washed successively, once with ethanol, twice with acetone, and twice with light petroleum ether and air-dried. LPS was extracted from the air-died cellular material by the hot phenol-water extraction procedure of Westphal and Jann (1965). LPS was obtained from the aqueous phase after extensive dialysis and lyophilization. H. pylori LPS from parental strains was further purified by ultracentrifugation and the pellet suspended in distilled water and lyophilized.

Preparation of core oligosaccharides LPS (25-30 mg) was hydrolyzed in 0.1M sodium acetate buffer, pH 4.2 for 2 h at 100° C.; the solution was cooled and the precipitated lipid A was removed by low-speed centrifugation. The supernatant solution was lyophilized and water soluble components were fractionated by gel filtration on a Bio-Gel P-2 column (1.6 cm×95 cm, 200-400 mesh, BioRad) equilibrated with pyridinium acetate (0.05 M, pH 4.5). Elution was performed with pyridinium acetate (0.05 M, pH 4.5). The fractions (1 mL) were monitored for neutral glycoses (Dubois et al. 1956) and those giving positive reaction were combined and lyophilized.

Analytical methods Glycoses were determined by GLC as their alditol acetate derivatives. Samples (0.2-0.5 mg) were hydrolyzed with 2M trifluoroacetic acid (TFA) for 16 h at 100° C. and evaporated to dryness under a stream of nitrogen. The liberated glycoses were reduced with sodium borohydride (NaBH₄) and acetylated (Ac₂O) as previously described (York et al. 1985). The configuration of peracetylated heptitol derivatives was determined to be L-glycero-D-manno or D-glycero-D-manno by comparison of their GLC retention times with that of an authentic standard. Hexoses were determined to have the D-configuration by GLC analysis of their acetylated (R)-2-octyl glycoside derivatives (Gerwig et al. 1979).

Methylation analysis was performed on lipopolysaccharide samples (1-3 mg) with iodomethane in dimethylsulfoxide containing an excess of sodium hydroxide (Ciucanu and Kerek, 1984) and permethylated alditol acetates were characterized by GLC-MS in the EI mode.

GLC-MS analysis was performed with a Saturn 2000 GC-MS system using J&W DB17-MS (0.25 mm ID×0.25 μm film thickness (df)×30 mL). Samples (0.1 to 5 μL) were injected depending on a sample concentration. The injector was held at 265° C. with a split ratio of 1:25. Oven temperature program started at 180° C. and was ramped at a rate of 3.5° C./min to 280° C. Helium carrier flow rate was held at 1.2 mL/min using electronic control. Mass spectrometer was held at 230° C. All experiments were performed in EI mode. All turning parameters were computer optimized daily and the retention times of alditol acetates were updated weekly. During the analysis, the spectrometer was set to scan from 40 to 650 m/z and the data analysis was performed on Saturn Workstation 5.4.

FAB-MS analysis in a positive mode was performed on permethylated LPS samples using a JEOL AX505H double focusing sector mass spectrometer. 6 kV Xenon atom was used to ionize the sample. The sample was typically mixed with a solution of 1:1 thioglycerol/glycerol, although thioglycerol and glycerol alone was also used for some samples.

Electrospray mass spectrometry Samples were analyzed on a crystal Model 310 CE instrument (ATI Unicam, Boston, Mass., USA) coupled to an API 3000 mass spectrometer (Perkin-Elmer/Sciex, Concord, Canada) via a microionspray interface. A sheath solution (isopropanol-methanol, 2:1) was delivered at a flow rate of 1 μL/min to a low dead volume tee (250 μm i.d., Chromatographic Specialities, Brockville, Canada). All aqueous solutions were filtered through a 0.45-μm filter (Millipore, Bedford, Mass., USA) before use. An electrospray stainless steel needle (27 gauge) was butted against the low dead volume tee and enabled the delivery of the sheath solution to the end of the capillary column. The separation were obtained on about 90 cm length bare fused-silica capillary using 10 mM ammonium acetate/ammonium hydroxide in deionized water, pH 9.0, containing 5% methanol. A voltage of 25 kV was typically applied at the injection. The outlet of the capillary was tapered to ca. 15 μm i.d. using a laser puller (Sutter Instruments, Novato, Calif., USA). Mass spectra were acquired with dwell times of 3.0 ms per step of 1 m/z unit in full-mass-scan mode. For CZE-ES-MS/MS experiments, about 30 nL sample was introduced using 300 mbar for 0.1 min. The MS/MS data were acquired with dwell times of 1.0 ms per step of 1 m/z unit. Fragment ions formed by collision activation of selected precursor ions with nitrogen in the RF-only quadrupole collision cell, were mass-analyzed by scanning the third quadrupole. Collision energies were typically 60 eV (laboratory frame of reference).

Mouse Colonization Mouse colonization was performed as described elsewhere by Logan et al. (2000). In an initial experiment, mice were inoculated by gavage twice 5 days apart with bacterial suspensions of approximately 10⁹ organisms and 10⁶ organisms respectively. In the second experiment the mice were given three inocula spread over 5 days with bacterial suspensions of approximately 11⁹ organisms per dose.

Preparation of fluorescently labeled bacteria Bacteria were grown in Brucella broth (Difco) with 10% FBS and harvested by centrifugation at 2,400×g for 5 min. The bacteria (1×10⁸ cells) were then resuspended into 2 mL of a freshly prepared 5- (and 6-) carboxyfluorescein diacetate, succinimidyl ester (CFDA-SE) (Molecular Probes, Oreg., USA) 5 μM solution and the reaction was carried out as described by Logan et al. (1998).

Cell culture HuTu-80 (ATCC HTB-40), derived from a human duodenal adenocarcinoma, was obtained from the American Type Culture Collection. It was maintained in tissue culture flasks as adherent monolayers in Minimal Essential Media (MEM) (Gibco) supplemented with 10% (vol/vol) FBS without antibiotics. For use in adherence assays, cells were trypsinized with 0.25% trypsin (Sigrna) (10 min, 37° C.), centrifuged at 200×g for 5 min, washed once with PBS and resuspended in PBS, pH 7.4 at a final concentration of 1×10⁶.

Adherence assay The fluorescently labeled bacteria were added to mammalian cells at a ratio of 100:1 and incubated at 37° C. for 30 min with shaking (150 rpm)(Dunn et al., 1991). Unbound bacteria were removed by centrifugation at 200×g for 5 min through a 15% (weight/vol) sucrose solution and the remaining cells were fixed with 3% (vol/vol) formaldehyde and analyzed by flow cytometry.

Flow cytometry Measurement of H. pylori adhering to epithelial cells was made with a Coulter EPIC XL flow cytometer (Coulter, Miami, Fla.) In total 10,000 ungated events were collected and the resulting histograms were produced. The percentage of mammalian cells with adherent bacteria was then determined comparing the histograms of the test samples to mammalian cells with unstained bacteria.

Results

Identification of putative H. pylori DD-heptosyl transferases Computer database searches of the genome of H. pylori 26695 were conducted using the BLAST search engine (Altschul, et al., 1997) for genes which showed structural homology to heptosyl-transferases. Several different heptosyl-transferases were used as the query sequences for these searches including the WaaC genes from E. coli, S. typhimurium, Campylobacter coli, Campylobacter jejune, Campylobacter hyoilei, and H. pylori 26695 (HP0279). One gene, HP0479, was identified through this process.

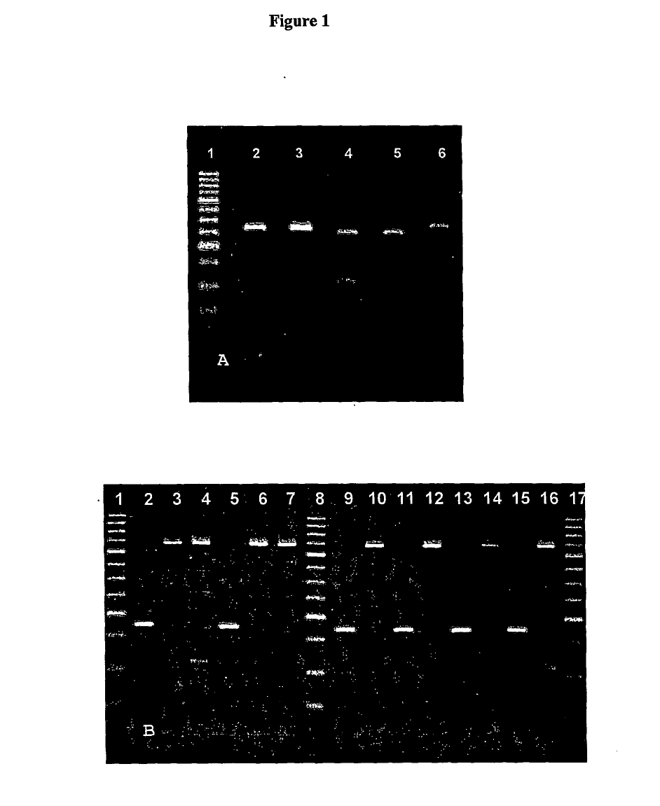

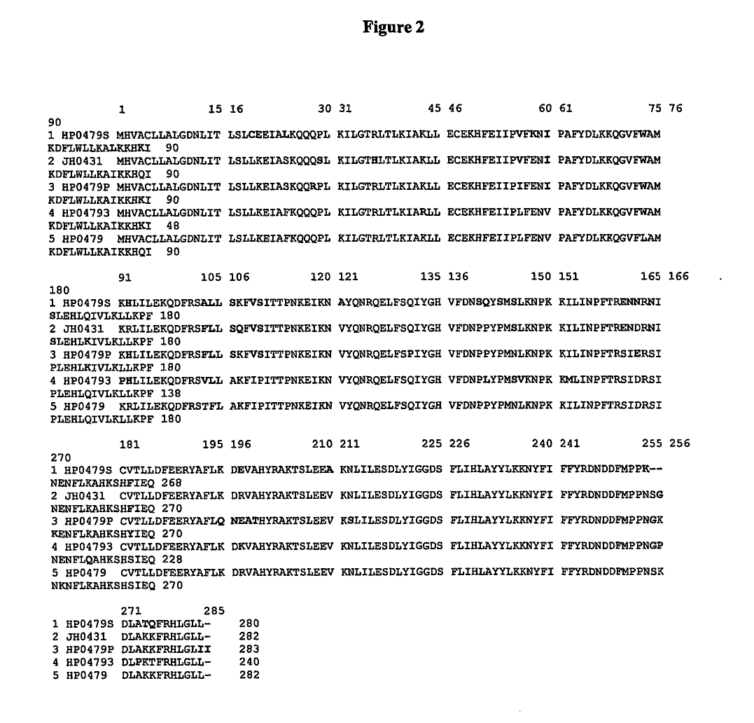

Cloning of HP0479 gene from H. pylori strains The primers used for PCR amplification, HP0479-F1 and HP0479-R1 (Table 2), were chosen from regions flanking the gene from the total genome sequence of H. pylori 26695. Primer HP0479-P1 starts 140 bp from the start of the BP0479 gene while primer HP0479-R1 ends 49 bp downstream of the gene. With these primers a PCR product of 1242 bp was expected. Amplification was carried out as described in Materials and Methods. A PCR product of approximately the correct size was amplified from DNA from H. pylori strains 26695, Sydney, ATCC 43504, PJ1, and strain 0:3 (FIG. 1, Panel A). The fragments were cloned into the SmaI site of pUC19 and transformed into E. coli DH5α. Standard blue/white selection was carried out and several clones were isolated. The orientation of the cloned fragments was determined by restriction endonuclease analyses. The clones were designated p0479G (from 26695 genome strain), p0479S (from Sydney strain), p04793 (from strain 0:3), p0479T (from the type strain ATCC43504) and p0479P (from PJ1). The DNA sequence of the HP0479 genes from p0479G, p0479S, p04793, and p0479P was determined. Amino acid sequences were predicted from the DNA sequence and these were aligned with the sequence of the HP0479 homolog from J99, JHP0431 (FIG. 2).

Mutagenesis of the cloned HP0479 gene PCR was used to generate a blunt-ended insertion site within the HP0479 gene of p0479G for insertion of the Kanamycin cassette. The primers used to generate the blunt-ended insertion site, HP0479-mutF1 and HP0479-mutR1, are shown on Table 2. The thermo-stable polymerase PwoI was used to amplify the fragment for insertion mutagenesis. The primers generated a 30 bp deletion in the middle of the HP0479 gene and the PCR product was expected to be 3837 bp in length (the gene+pUC19). The 1398 bp SmaI fragment from plasmid pIP1433, which harbored a Campylobacter coli Kanamycin resistance marker, was cloned into the PCR generated insertion site. The resulting ligation mix was transformed into E. coli DH5α. Candidate clones were selected on media containing Kanamycin. Plasmid DNA was isolated from Kanamycin resistant clones and the presence of the Kanamycin cassette was confirmed by restriction endonuclease analysis and agarose gel electrophoresis (data not shown). One plasmid, p0479GM1, was used for transformation of H. pylori.

Transformation of H. pylori with the mutated HP0479 gene Since the pUC19 plasmid was not compatible with H. pylori, it acted as a suitable suicide vector for the transfer of the mutated HP0479 gene into H. pylori strains. Selection for Kanamycin resistance was used to isolate H. pylori that had incorporated the mutated HP0479 gene. The HP0479 mutant gene was introduced into several strains of H. pylori using this method. The H. pylori strains mutagenized were strain 26695, Sydney strain, strain 0:3, Type strain (ATCC 43504), and strain PJ1. Kanamycin resistant transformants were obtained for all strains. Chromosomal DNA was isolated from all of the Kanamycin resistant transformant strains and the insertion of the HP0479 mutant was confirmed by PCR. It was possible that the location of the HP0479 homolog in the other strains could vary compared to 26695. Therefore, internal PCR primers to the start and end of the HP0479 gene were used for confirming the insertion of the Kanamycin cassette (Table 2, primers HP0479-GF1, HP0479-GR1). These primers amplify a 909 bp fragment from H. pylori 26695 DNA and also amplified a fragment of similar size from all of the other strains mentioned above (FIG. 1, Panel B). If the mutated HP0479 gene had been incorporated, the size of this PCR product was expected to shift to 2217 bp. In all strains, the incorporation of the mutant HP0479 gene was confirmed (FIG. 1, Panel B). Membrane preparations of the mutants and the parental strain showed that the mutation of the HP0479 gene did not alter the membrane protein profile (data not shown). The mutant H. pylori strains were designated H. pylori 0479GM1 (26695 mutant), H. pylori 0479SM1 (Sydney strain mutant), H. pylori 04793M1 (0:3 mutant), H. pylori 0479TM1 (ATCC 43504 type strain mutant) and H. pylori 0479PM1 (PJ1 mutant).

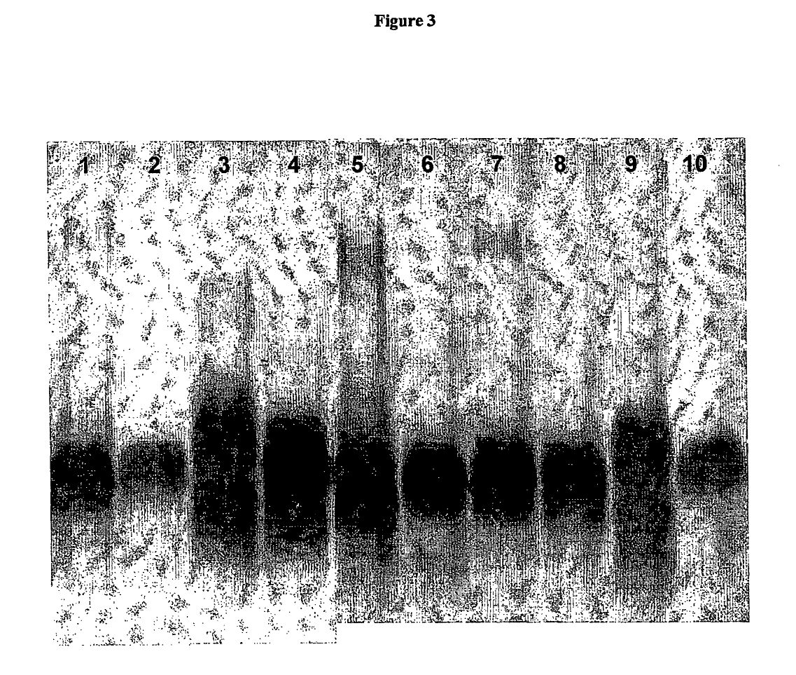

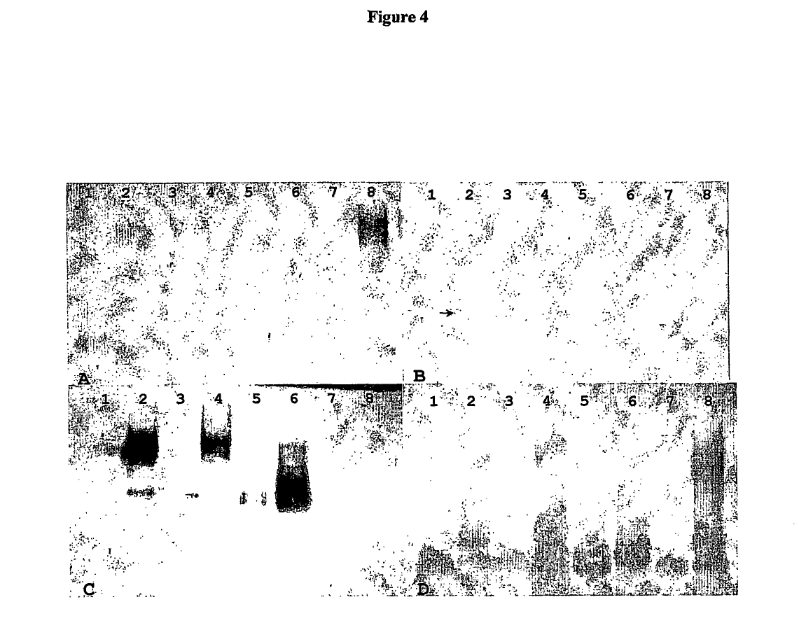

Analysis of the LPS of H. pylori strains by SDS-PAGE The LPS profiles of both the parental and HP0479 mutant strains were analyzed by SDS-PAGE of whole cell LPS preparations using methods modified from Hitchcock and Brown (1983). In all cases, the mutation of the HP0479 gene caused an alteration in the core and the loss of the O-antigen (FIG. 3). Western blots of LPS samples from mutant and parental strains were also prepared and probed with monoclonal antibodies raised against Lewis X and Lewis Y blood group antigens, as well as with a polyclonal antiserum raised against H. pylori PJ1 (FIG. 4). The Lewis X antibodies reacted against the parental 26695 and weakly against the parental 0:3 strain LPS. The Lewis Y antibody reacted strongly against the parental strain LPS of 0:3, 26695 and Sydney strain. The PJ1 polyclonal antibody has been shown previously to cross react with all of the H. pylori strains tested and was used as a positive control. As expected this antiserum reacted with the LPS of all of the strains, both mutant and parental (FIG. 4).

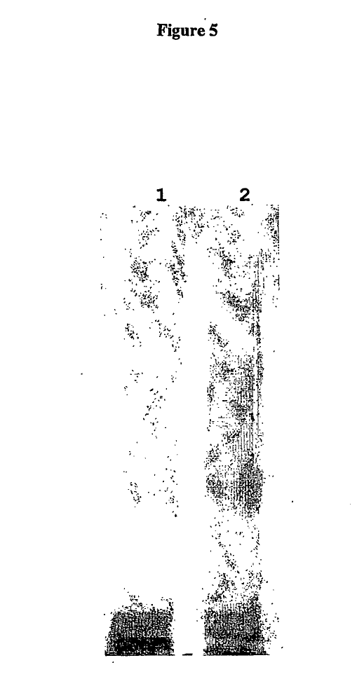

Mutagenesis of HP0480 In order to determine any polar effects that may occur as the result of mutagenesis of the HP0479 gene, the gene immediately downstream of HP0479, HP0480, was mutagenized. The HP0480 gene was amplified as described in Materials and Methods, cloned into pUC19 and mutagenized by the insertion of a C. coli Chloramphenicol cassette into a unique SmaI site. This mutant plasmid was then transformed into H. pylori 26695 and Sydney strain. Chloramphenicol resistant strains were isolated and whole cell LPS samples were run on SDS-PAGE. FIG. 5 shows that the LPS profiles on SDS-PAGE of the parental and the 0480SM1 H. pylori Sydney strains are identical.

Structural characterization of H. pylori LPS mutants 0479GM1, 0479SM1 and 04793M1 Sugar analysis of the HP0479 LPS mutants indicated reduction in the amount of O-chain components, namely L-Fuc, D-Gal, D-GlcNAc, and DD-Hep (Table 3) as compared with parent LPS. Methylation analysis of the intact LPS from each strain indicated absence of 3-substituted and 6-substituted D-Glc, 3-substituted DD-Hep (for H. pylori 04793M1) and 6-substituted DD-Hep (for H. pylori 04793M1 and H. pylori 0479GM1 LPS) and a significant decrease in 2-substituted DD-Hep as compared to the intact LPS from corresponding parental strains, suggesting deficiencies in the core biosynthesis.

FAB-MS analysis in the positive mode of the permethylated LPS from each strain indicated the presence of primary glycosyl oxonium ions at m/z 260 [GlcNAc]⁺ and m/z 434 [Fuc,GlcNAc]⁺ and secondary glycosyl oxonium ions at m/z 228 (260-32) [GlcNAc]⁺ and m/z 402 (434-32) [Fuc,GlcNAc]⁺. This evidence together with the absence of the primary glycosyl oxonium ion at m/z 682 [Fuc, GlcNAc, Hep]⁺ suggested that the mutant LPS structure was lacking DD-Hep residue which bridges O-chain and the core oligosaccharide in the respective parent LPS (Monteiro et al., 2000, Logan et al., 2000).

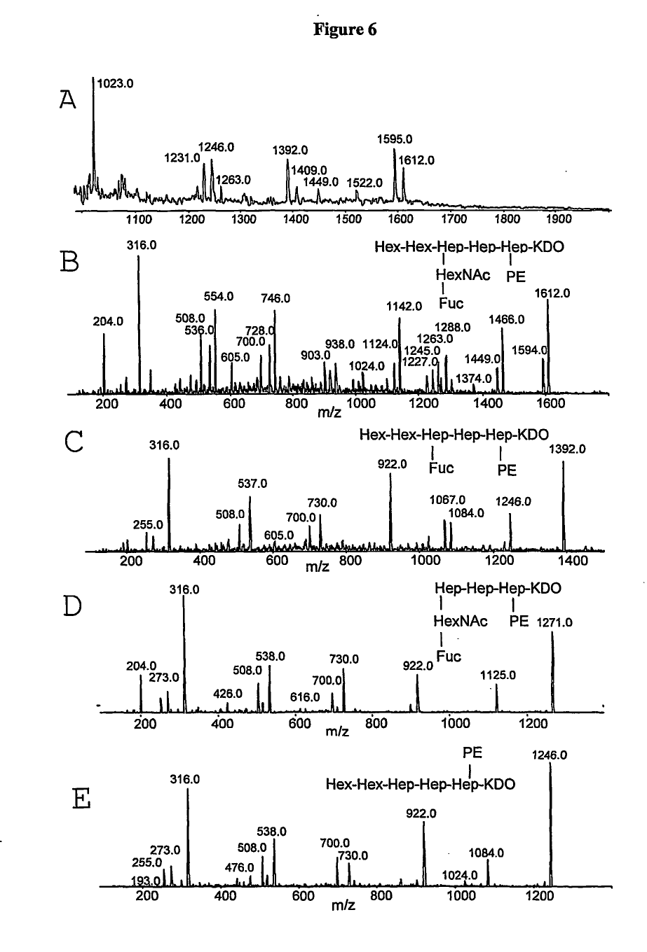

LPS from H. pylori 0479SM1 and 26695 was delipidated and desalted following gel filtration chromatography on a Bio-Gel P-2 column. Fractions containing core oligosaccharide components were subjected to the mass spectrometric analysis using combined capillary zone electrophoresis-electrospray-mass spectrometry (CZE-ES-MS) in the positive mode, followed by MS/MS analysis of the most abundant oligosaccharide fragments. The product ion spectrum showed singly charged fragment ions at m/z 1612 and m/z 1594, containing an anhydro-KDO. These fragment ions could be assigned to a glycoform HexHexHep(HexNAcFuc)HepHep(PE)KDO (FIG. 6), based on the linkage and FAB-MS analyses data and recent structural studies (Monteiro et al. 2000). Further structural evidence was obtained in a MS/MS experiment where the singly charged ion at m/z 1392 was selected as a precursor. Observation of the diagnostic ion at m/z 1246, arising from the loss of Fuc, indicated the structure of the precursor ion m/z 1392 to be HexHexHep(Fuc)HepHep(PE)KDO. The MS/MS spectrum of m/z 1246 was consistent with the core fragment HexHexHepHepHep(PE)KDO as confirmed by a consecutive cleavage of glycosidic bonds yielding a direct sequence assignment (Table 4). These structural assignments are consistent with the presence of 2,7-substituted DD-Hep, 7-substituted DD-Hep and 2-substituted DD-Hep in the methylation analysis of LPS mutants H. pylori 0479GM1, H. pylori 0479SM1, H. pylori 04793M1. Absence of the second DD-heptose residue (DDHepII) which serves as a link between the O-chain and the core oligosaccharide and is glycosylated by α-1,6-glucan, resulted in the loss of O-chain and DD-heptan (for serotype 0:3). A comparison of the H. pylori LPS structures from mutant and parental strains is presented in FIG. 7.

Mouse Colonization studies The effects on colonization were investigated using the H. pylori Sydney strain. This strain has been shown to consistently colonize mice and has been universally used as a mouse colonization model for H. pylori (Lee et al., 1997; Ferrero et al., 1998; Conlan, et al., 1999; Logan et al., 2000). In this study two separate trials were carried out. In each trial 10 mice were given H. pylori Sydney strain wild type cultures and 10 were given H. pylori 479SM1.

In the first trial H. pylori was administered twice orogastrically 5 days apart with approximately 10⁶ to 10⁸ bacteria as determined by plate counts of the inoculum. One week and 12 weeks later, 5 mice from each group were sacrificed, their stomachs homogenized, and plate counts were performed to determine the extent of colonization. In the second trial, H. pylori was administered three times over a 5 day period with approximately 1×10⁹ doses of either organism. In this trial the mice were sacrificed two weeks and four weeks after bacterial inoculation. In both trials the parental strain was able to establish colonization which persisted at significant levels even after 12 weeks (Tables 5 and 6). In contrast, the mutant strain was never detected in the stomachs of any of the mice challenged with it (Tables 5 and 6). To confirm the absence of the mutant strain, PCR was carried out on the stomach homogenates using the HP0479-GF1 and HP0479-GR1 primers. The expected PCR products were obtained from the mice inoculated with the H. pylori SS1, but not from the stomach homogenates of mice inoculated with mutant strain (data not shown).

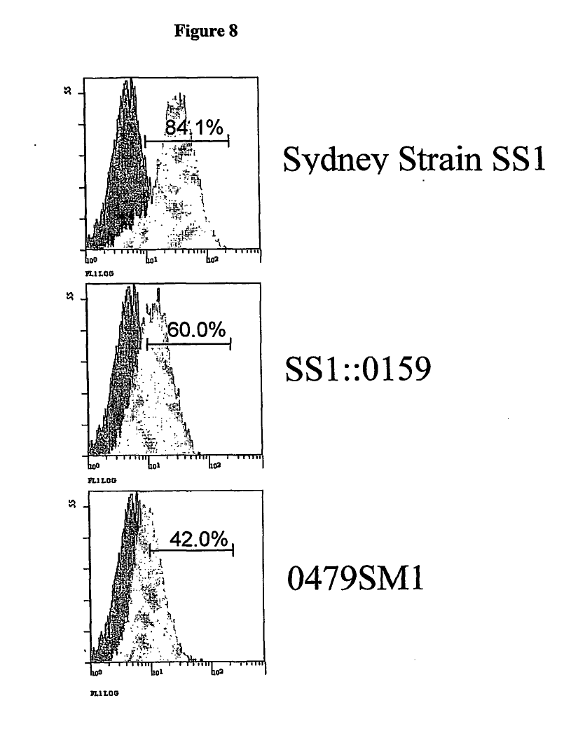

Flow cytometric analysis of the adhesion of H. pylori Following the incubation of HuTu-80 cells with the fluorescently labeled bacteria for 30 min and subsequent analysis by flow cytometry, levels of adhesion of the H. pylori parental strain SS1, mutant strain SS1::HP0159 and mutant strain 0479SM1 to HuTu-80 cells were compared. The rates of H. pylori adherence to HuTu-80 cells were 84.1, 60.0 and 42.0%, respectively, indicating that H. pylori adherence to HuTu-80 cells was affected by the absence of the LPS O-chain and the degree of truncation in the LPS molecule (FIG. 8).

Discussion

Lipopolysaccharides are the main surface antigens of Gram-negative bacteria, and are essential for the physical integrity and function of the bacterial outer membrane. Despite the importance of LPS in bacterial pathogenesis, H. pylori LPS has received limited attention and its' role in the pathogenic process has not been clearly established. Similar to LPS from other species, the basic structure of H. pylori LPS consists of three distinct regions: O-chain polysaccharide composed of repeating units, covalently linked to a core oligosaccharide, which in turn is attached to a hydrophobic lipid A moiety (Raetz et al., 1990). The structure of H. pylori LPS is unique among Gram-negative bacteria having four consecutive heptosyl residues in the core region (two D-glycero-D-manno-heptose (DD-heptose) and two L-glycero-D-manno-heptose (LD-heptose) residues). The core region also includes a trisaccharide moiety branching from DDHepI (FIG. 7)(Aspinall and Monteiro, 1996; Aspinall et al., 1996; Monteiro et al., 1998a; Monteiro et al., 2000b). This core structure is conserved among H. pylori species and no structural variability in this region has been reported so far. Some variability is observed in the presence or absence of a polyglucan side chain on DDHepII and the presence of either mono-ester phosphate or 2-phosphoethanolamine on HepI (FIG. 7) (Aspinall and Monteiro, 1996; Aspinall et al., 1996; Monteiro et al., 1998a; Monteiro, et al, 2000a). The O-antigen extends from DDHepII or in some strains is linked to the core through a heptan region composed of a α(1-3)-linked DD-heptose polymer that is extended by additional 2- and 6-linked DD-Hep residues. The length of this heptan region may vary from strain to strain (Aspinall et al., 1997; Monteiro, et al., 2000b) and in some strains O-chain was found to be directly linked to the 0-2 position of DDHepII. The length of the O-chain polysaccharide varies considerably from strain to strain and phase variation in the population of a given strain has been reported (Monteiro, et al., 2000a; Monteiro, et al., 2000b;, Appelmelk et al., 1998; Aspinall et al., 1997). This ability is thought to contribute to pathogenicity by allowing the organism to survive and adapt to various environments (Appelmelk et al., 1998).

A great deal of attention has been given to the O-chain polysaccharide of H. pylori due to the expression of Lewis antigens structurally related to the determinants of the human ABH blood group system, in particular the type 2 Lewis X and Lewis Y blood group antigens. Recently Monteiro et al. (2000a) have found that H. pylori isolated from Asian patients expressed predominantly type 1 Lewis antigens in their LPS. The role of the O-antigen in pathogenesis remains unclear. It has been hypothesized that the Lewis antigens aid the organism in evading the host immune system by mimicking the host blood group antigens, however Taylor et al., (1998) did not find a correlation between the blood antigen expressed by the H. pylori with the antigen expressed by the patient. It has also been suggested that inflammation may be related to autoimmune reactions to the Lewis antigens (Appelmelk, et al., 1997).

A study by Logan et al. in 2000, showed that mutation of a gene encoding β-1,4 galactosyl transferase (HP0826), resulted in a truncated O-antigen and reduction in the ability of H. pylori to colonize the murine stomach. Edwards et al. (2000) showed that Lewis X components on the H. pylori O-antigen promoted adhesion to gastric epithelial cells in vitro. Thus it is likely that the O-antigen contributes to pathogenesis as a colonization factor.

As mentioned, the core of H. pylori LPS is a unique combination of both DD and LD forms of heptose. In addition some strains further extend this polyheptose region by the addition of DD-heptose between the core and the O-antigen (FIG. 7)(Aspinall et al., 1996; Aspinall and Monteiro 1996; Aspinall et al., 1997). To date none of the transferases involved in the biosynthesis of the core or the addition of DD-heptose to the heptan-linking region have been functionally identified. From the standpoint of developing therapeutic intervention strategies against H. pylori, this region is of particular interest as it provides a fairly constant target for H. pylori without the variation that is seen in the O-antigen.

Several organisms, including enteric bacteria and H. pylori, incorporate L-glycero-α-D-manno-heptose (LD-heptose) into the core of their LPS. There are few examples of other organisms that incorporate D-glycero-α-D-manno-heptose (DD-heptose) into their LPS (Süsskind, et al., 1998; Stevens, et al., 1997; Toman, R. and L. _kultéty, 1995). Since LD-heptose and DD-heptose are structurally related, differing stereochemically at the glycero moiety of the molecule, some homology may exist between LD- and DD-heptosyl transferases. Using amino acid sequences of heptosyl transferase proteins from other organisms, computer searches were used to identify proteins in H. pylori that could be potential DD-heptosyl transferases. Based on similarity to other heptosyl-transferases, the HP0479 gene of H. pylori 26695 was identified as a putative heptosyl-transferase.

ClustalW Multiple Sequence alignments (Higgins, et al., 1992; Thompson et al., 1994) of HP0479 against the amino acid sequences of several other heptosyl transferases showed that HP0479 had significant homology in conserved regions with the other transferases. According to the classification of glycosyl transferase families as described by Campbell et al. (1997) HP0479 belongs to family 9, which includes heptosyl-transferases.

Mutational analysis of BP0479 showed that H. pylori strains carrying mutations of this gene had truncated LPS. Western blots of whole cell LPS samples immunoblotted against Lewis antigens also showed a loss of O-antigen. The LPS profiles of all of the mutant strains of H. pylori tested were the same. This showed that HP0479 did not encode a heptosyl transferase involved in the synthesis of the heptan linker region that is present in the 26695 and 0:3 strains, but not present in Sydney strain. Polar mutational effects were ruled out as mutations to the gene immediately downstream of HP0479, HP0480, produced wild type LPS. Structural analysis of the mutant strains confirmed that the LPS was truncated at DDHepI (FIG. 7, Table 4), showing that HP0479 was indeed responsible for the α(1,2)-DD-heptosyltransferase activity which adds DDHepII to the LPS core structure.

In the earlier study by Logan et al. (2000), it was shown that the loss of O-antigen reduced the amount of colonization by H. pylori in mice. Here mutations of HP0479 reduced colonization even further and perhaps abolished colonization all together, as no detectable colonization was observed in mice inoculated with the HP0479 mutant Sydney strain. Interestingly, the HP0826 mutant reported by Logan et al. (2000) reduced, but did not abolish colonization. Structurally the HP0826 and the HP0479 H. pylori SS1 mutants differ by two sugar residues. It is possible that colonization requires some minimal length of LPS.

In summary, HP0479 is the first DD-heptosyltransferase from H. pylori that has been functionally identified. Structural data from several other strains of H. pylori (Aspinall et al., 1996; Aspinall and Monteiro 1996; Aspinall et al., 1997; Monteiro, et al. 2000b) show that there may potentially be up to 7 or more heptosyl-transferase genes involved in the assembly of H. pylori LPS. The heptan regions of the LPS in H. pylori, particularly the heptoses in the core, present a more constant target for possible therapeutic interventions than the more variable O-antigen. Evidence presented here indicates that LPS may be an important colonization factor in H. pylori pathogenesis.

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. All publications, patents and patent applications mentioned herein are incorporated herein by reference for the purpose of describing and disclosing the cell lines, vectors, methodologies etc. which are reported therein which might be used in connection with the invention. Nothing herein is to be construed as an admission that the invention is not entitled to antedate such disclosure by virtue of prior invention.

It must be noted that as used herein and in the appended claims, the singular forms “a”, “an”, and “the” include plural reference unless the context clearly dictates otherwise. Thus, for example, reference to “a host cell” includes a plurality of such host cells, reference to the “antibody” is a reference to one or more antibodies and equivalents thereof known to those skilled in the art, and so forth.

TABLE 1
Bacterial strains used in Example 1

| Bacterial Strains | Reference or source |
|---|---|
| Helicobacter pylori 26695 | Tombs, et al. 1997 |
| Helicobacter pylori PJ1 | Clinical isolate |
| Helicobacter pylori serogroup 0:3 | Aspinall, et al., 1997 |
| Helicobacter pylori ATCC 43504 | American Type Culture Collection |
| Helicobacter pylon Sydney (SS1) | Lee, et al. 1997 |
| Escherichia coli DH5α | BRL |
| Helicobacter pylori 0479GM1 | This study |
| Helicobacter pylori 0479SM1 | This study |
| Helicobacter pylori 04793M1 | This study |
| Helicobacter pylori 0479TM1 | This study |
| Helicobacter pylori 0479PM1 | This study |
| Helicobacter pylori 0480SM1 | This study |

TABLE 2
Primers used for PCR amplification

| | | annealing |
|---|---|---|
| Primer | primer sequence(5′->3′) | temperature |
| HP0479-F1 | GCCTTTATCAAGCTAGAG | 60° C. |
| HP0479-R1 | CATAAATGTCCTAACAAGC | 60° C. |
| HP0479-mutF1 | CAAAACCGCCAGGAGTTG | 55° C. |
| HP0479-mutR1 | GGTTATGGGAATGAATTTGG | 55° C. |
| HP0479-GF1 | ATGCATGTTGCTTGTCTTTTGG | 58° C. |
| HP0479-GR1 | TTATAATAGCCCCAAATGGC | 58° C. |
| HP0480-F1 | GATAACCTCATCACGCTTAG | 60° C. |
| HP0480-R1 | TTCAATCCATTCTAACGC | 60° C. |

TABLE 3
Approximate molar ratios of the alditol acetate derivatives of
HP0479 isogenic mutants intact LPS (numbers in parentheses
indicate ratios obtained for respective parent strains, analyses
were performed on broth grown cells).

| Strain | L-Fuc | D-Glc | D-Gal | D-GlcNAc | DD-Hep | LD-Hep |
|---|---|---|---|---|---|---|
| H. pylori 0479GM1^{b} | 0.9 (4) | 3.0 (9) | 1.4 (9) | 4.2 (15) | 0.9 (5.5) | 1.0 (1) |
| H. pylori 0479SM1^{a} | 1.0 (3.5) | 1.1 (4) | 1.1 (12) | 4.0 (9.2) | 0.9 (2.5) | 1.0 (1) |
| H. pylori 04793M1^{b} | 0.9 (8) | 1.3 (7) | 1.0 (12) | 2.0 (13) | 0.9 (6) | 1.0 (1) |

^{a}fermenter grown cells
^{b}broth grown cells

TABLE 4
Positive ion CE-ES-MS data and proposed structures for H. pylori 0479
LPS mutants.

| Molecular mass | | |
|---|---|---|
| observed | calculated^{a} | Proposed structure |
| 1611 (1593)^{b} | 1611.3 | 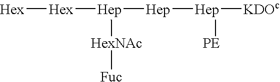 |
| 1391^{b} | 1390.0 | 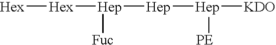 |
| 1270^{b} | 1269.0 | 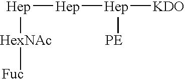 |
| 1245^{b} | 1244.0 | 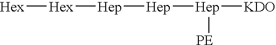 |

^{a}Average mass units were used for calculation of molecular mass values based on proposed composition as follows: Hex, 162.15; HexNAc, 203.20; Fuc, 146.14; Hep, 192.17; KDO, 220.18; PEA, 123.05; H₂O, 18.02;
^{b}observed fragment ion corresponds to anhydro-KDO-containing glycoform;
^{c}the most abundant glycoform (based on the fragment ion intensity).

TABLE 5
Viable counts of H. pylori Sydney strain
and HP0479SM1 recovered from mice in Trial 1.

| | | Log₁₀ CFU |
|---|---|---|
| Weeks post-inoculation | Inoculum | (x from 5 mice) |
| Week 1 | H. pylori SS1 | x = −4.758 ± 0.93 |
| | H. pylori HP0479SM1 | BDL |
| Week 12 | H. pylori SS1 | x = −5.02 ± 1.060 |
| | H. pylori HP0479SM1 | BDL |

BDL = below detectable limits

TABLE 6
Viable counts of H. pylori Sydney strain
and HP0479SM1 recovered from mice in Trial 2.

| | | Log₁₀ CFU |
|---|---|---|
| Weeks post-inoculation | Inoculum | (x from 5 mice) |
| Week 2 | H. pylori SS1 | x = −5.34 ± 0.70 |
| | H. pylori SS1 HP0479M1 | BDL |
| Week 4 | H. pylori SS1 | x = −5.82 ± 0.12 |
| | H. pylori SS1 HP0479M1 | BDL |

### References

1. Aim, R. A., L -S. L. Ling, D. T. Moir, B. L. King, E. D. Brown, P. C. Doig, D. R. Smith, B. Noonan, B. C. Guild, B. L. dejonge, G. Carmel, P. J. Tummino, A. Caruso, M. Uria-Nickelsen, D. M. Mills, C. Ives,. R. Gibson, D. Merberg, S. D. Mills, Q. Jiang, D. E. Taylor, G. F. Vovis, and T. J. Trust. 1999. Genomic-sequence comparison of two unrelated isolates of the human gastric pathogen Helicobacter pylori. Nature 397(6715):176-180.

2. Altschul, S. F., T. L. Madden, A. A. Schäffer, J. Zhang, Z. Zhang, W. Miller, and D. J. Lipman. 1997. Gapped BLAST and PSI-BLAST: a new generation of protein database search programs. Nucl. Acids Res. 25:3389-3402.

3. Appelmelk, B. J., R. Negrini, A. P. Moran, and E. J. Kuipers. 1997. Molecular mimicry between Helicobacter pylori and host Trends Microbiol. 5:70-73.

4. Appelmelk, B. J., B. Shiberu, C. Tinks, N. Taps, P. Y. Zheng, T. Verboom, J. Maaskant, C. H. Hokke, W. E. C. M. Schiphorst, D. Blanchard, I. M. Simoons-Smit, D. H. Van Den Eijnden, and C. M. J. E. Vandenbroucke-Grauls. 1998. Phase variation in Helicobacter pylori lipopolysaccharide. Infect. Immunol. 66(1):70-76.

5. Aspinall, G. O. and M. A. Monteiro. 1996. Lipopolysaccharides of Helicobacter pylori strains P499 and MO19: structures of the O-antigen and core oligosaccharide regions. Biochemistry 35:2498-2504.

6. Aspinall, G. O., M. A. Monteiro, H. Pang, E. J. Walsh, and A. P. Moran. 1996. Lipopolysaccharide of the Helicobacter pylori type strain NCTC 11637 (ATCC43504): structure of the O-antigen chain and core oligosaccharide regions. Biochemistry 35:2489-2497

7. Aspinall, G. O., M. A. Monteiro, R. T. Shaver, L. A. Kurjanczyk, and J. L. Penner. 1997. Lipopolysaccharide of Helicobacter pylori serogroups 0:3 and 0:6. Structures of a class of lipopolysaccharides with reference to the location of oligomeric units of D-glycero-α-D-manno-heptose residues. Eur. J. Biochem. 248:592-601.

8. Blaser, M. J. 1990. Helicobacter pylori and the pathogenesis of gastroduodenal inflammation. J. Infect. Dis. 161:626-633.

9. Blaser, M. J. 1995. The role of Helicobacter pylori in gastritis and its progression to peptic ulcer disease. Aliment. Pharmacol. Ther. 9:27-30.

10. Campbell, J. A., G. J. Davies, V. Bulone, and B. Henrissat. 1997. A classification of nucleotide-diphospho-sugar glycoslytransferases based on amino acid sequence similarities. Biochem. J. 326: 929-939.

11. Chung, C. T., and R. H. Miller. 1988. A rapid and convenient method for preparation and storage of competent bacterial cells. Nucl. Acids Res. 16(8):3580.

12. Ciucanu, I. and F. Kerek. 1984. A simple and rapid method for the permethylation of carbohydrates. Carbohydr. Res. 131: 209-217.

13. Conlan, J. W., R. Kuolee, A. Webb, and M. B. Perry. 1999 Immunosuppression by a corticosteroid fails to exacerbate Helicobacter pylori infection in a mouse model of gastric colonisation. Can. J. Microbiol. 45:975-980.

14. Dubois, M., K. A. Gilles, J. K. Hamilton, P. A. Rebers, and F. Smith. 1956. Colorimetric method for determination of sugars and related substances. Anal. Chem. 28:350-356.

15. Dunn, B. E., M. Altmann, and G. P. Campbell. 1991. Adherence of Helicobacter to gastric carcinoma cells: analysis by flow cytometry. Rev. Infect. Dis. 13:S657-S664.

16. Dunn, B. E., H. Cohen, and M. J. Blaser. 1997. Helicobacter pylori. Clin. Microbiol. Rev. 10(4):720-741.

17. Edwards, N. J., M. A. Monteiro, G. Faller, E. J. Walsh, A. P. Moran, I. S. Roberts, and N. J. High. 2000. Lewis X structures in the O-antigen side-chain promote adhesion of Helicobacter pylori to the gastric epithelium. Mol. Microbiol. 35(6):1530-1539.

18. Ferrero, R. L., J -M. Thiberge, M. Huerre, and A. Labigne. 1998. Immune response of specific-pathogen-free mice to chronic Helicobacter pylori (strain SS1) infection. Infect. Immun. 66:1349-1355.

19. Gerwig, G. J., J. P. Kamerling, and J. F. G. Vliegenthart. 1979. Determination of the absolute configuration of the monosaccharides in complex carbohydrates by capillary G.L.C. Carbohydr. Res. 77:1-7.

20. Graham, D. Y. 1991. Helicobacter pylori: its epidemiology and its role in duodenal ulcer disease. J. Gastroenterol. Hepatol. 6:105-113.

21. Haas, R., T. F. Meyer, and J. P. M. Van Puten. 1993. A flagellated mutants of Helicobacter pylori generated by genetic transformation of naturally competent strains using transposon shuttle mutagenesis. Mol. Microbiol. 8:753-760.

22. Higgins, D. G., A. J. Bleasby, and R. Fuchs. 1992. CLUSTAL V: improved software for multiple sequence alignment Comput Appl. Biosci. 8(2):189-91.

23. Hitchcock, P. J., and T. M. Brown. 1983. Morphological heterogeneity among Salmonella lipopolysaccharide chemotypes in silver-stained polyacrylamide gels. J. Bacteriol. 154(1):269-277.

24. Jiang, Q., K. Hiratsuka, and D. E. Taylor. Variability in gene order in different Helicobacter pylori strains contributes to genome diversity. Mol. Microbiol. 20:833-842.

25. Karlsson, K. 2000. The human gastric colonizer Helicobacter pylori: a challenge for host-parasite glycobiology. Glycobiology 10(8):761-771.

26. Kidd, M., K. Miu, L. H. Tang, G. I. Pérez-Pérez, M. J. Blaser, A. Sandor, and I. M. Modlin. 1997. Helicobacter pylori lipopolysaccharide stimulates histamine release and DNA synthesis in rat enterochromaffin-like cells. Gastroenterology 113:1110-1117.

27. Labigne-Roussel, A., P. Courcoux, and L. Tompkins. 1988. Gene disruption and replacement as a feasible approach for mutagenesis of Campylobacter jejuni. J. Bacteriol. 170(4):1704-1708.

28. Laemmli, U. K. 1970. Cleavage of structural proteins during the assembly of the head of the bacteriophage T4. Nature 227:680-685.

29. Lee, A., J. O'Rourke, M. Corazon de Ungria, B. Robertson, G. Daskaolopoulos, and M. F. Dixon. 1997. A standardised mouse model of Helicobacter pylori infection: introducing the Sydney strain. Gastroenterology 112:1386-1397.

30. Logan, R. P. H., A. Robins, G. A. Turner, A. Cockayne, S. P. Boriello, and C. J. Hackeye. 1998. A novel flow cytometric assay for quantitating adherence of Helicobacter pylori to gastric epithelial cells. J. Immunol. Meth. 213: 19-30.

31. Logan, S. M., and T. J. Trust. 1984. Structural and antigenic heterogeneity of lipopolysaccharides of Campylobacter jejuni and Campylobacter coli. Infect. Immun. 45:210-216.

32. Logan, S. M., J. W. Conlon, M. A. Monteiro, W. W. Wakarchuk, and E. Altman. 2000. Functional genomics of Helicobacter pylori: Identification of a β-1,4 galactosyltransferase and generation of mutants with altered lipopolysaccharide. Mol. Microb. 35(5): 1156-1167.

33. Monteiro, M. A., K. H. Chan, D. Rasko, D. E. Taylor, P. Y. Zheng, B. J. Appelmelk, H. P. Wirth, M. Yang, M. J. Blaser, S. O. Hynes A. P. Moran, and M. B. Perry. 1998a. Simultaneous expression of type 1 and type 2 Lewis blood group antigens by Helicobacter pylori lipopolysaccharides. Molecular mimicry between H. pylori lipopolysaccharides and human gastric epithelial cell surface glycoforms. J. Biol. Chem. 273:11533-11543.

34. Monteiro, M. A., D. Rasko, D. E. Taylor, and M. B. Perry. 1998b. Glucosylated N-acetyllactosamine O-antigen in the lipopolysaccharide from Helicobacter pylori strain UA861. Glycobiology 8:107-112.

35. Monteiro, M. A., P. Zheng, B W Ho, S. Yokota, K. Amano, Z. Pan, D. E. Berg, K. H. Chan, L. L. MacLean, and M. B. Perry. 2000a. Expression of histo-blood group antigens by lipopolysaccharides of Helicobacter pylori strains from Asian hosts: the propensity to express type 1 blood-group antigens. Glycobiology 10(7):701-713.

36. Monteiro, M. A., B. J. Appelmelk, D. A. Rasko, A. P. Moran, S. O. Hynes, L. L. MacLean, K. H. Chan, F. St. Michael, S M. Logan, J. O'Rourke, A. Lee, D. E. Taylor and M. B. Perry. 2000b. Lipopolysaccharide structures of Helicobacter pylori genomic strains 26695 and J99, mouse model H. pylori Sydney strain, H. pylori P446 carrying sialyl Lewis X and H. pylori UA915 expression Lewis B. Eur. J. Biochem. 267:305-320.

37. Ootsubo, C., T. Okumura, N. Takahashi, H. Wakebe, K. Imagawa, M. Kikuchi and Y. Kohgo. 1997. Helicobacter pylori lipopolysaccharide inhibits acid secretion in pylorus-ligated conscious rats. Biochem Biophys. Res. Commun. 236:532-537.

38. Okumura T., E. Shoji, N. Takahashi, H. Wakebe, K. Imagawa, M. Kikuchi and Y. Kohgo. 1998. Delayed gastric emptying by Helicobacter pylori lipopolysaccharide in conscious rats. Dig. Dis. Sci. 43:90-94.

39. Parsonnet, J., S. Hansen, L. Rodriguez, A. B. Gelb, R. A. Warnke, E. Jellum, N. Orentreich, J. H. Vogelman, and G. D. Friedman. 1994. Helicobacter pylori infection and gastric lymphoma N. Engl. J. Med. 330(18):1267-71.

40. Parsonnet, J. 1995. Incidence of Helicobacter pylori infection. Aliment. Pharmacol. Ther. 9(Suppl 2):45-51.

41. Piotrowski, J., E. Piotorowski, D. Skrodzka, A. Slomiany, and B. L. Slomiany. 1997a. Induction of acute gastritis and epithelial apoptosis by Helicobacter pylori lipopolysaccharide. Scan J Gastroenterology 32:203-211.

42. Piotrowski, J., D. Skrodzka, A. Slomiany, and B. L. Slomiany. 1997b. Reversal of gastric somatostatin receptor inhibition by Helicobacter pylori lipopolysaccharide with ebrotidine and sulglycotide. Gen. Pharmacol. 28:705-708.

43. Raetz, C. R. H. 1990. Biochemistry of endotoxins. Annu. Rev. Biochem. 59:129-170.

44. Sakagami, T., J. Vella, M. F. Dixon. J. O'Rourke, R. Radcliffe, P. Sutton. 1997. The endotoxin of Helicobacter pylori is a modulator of host-dependent gastritis. Infect. Immun. 65:3462-3464.

45. Smith, R F. and T F. Smith. 1990. Automatic generation of primary sequence patterns from sets of related protein sequences. Proc. Natl. Acad. Sci. 87:118-122.

46. Smith, R. F. and T. F. Smith. 1992. Pattern-Induced Multi-sequence Alignment (PIMA) algorithm employing secondary structure-dependent gap penalties for comparative protein modeling. Protein Engineering 5:35-41.

47. Stevens, M. K., J. Klesney-Tait, S. Lumbley, K. A. Walters, A. M. Joffe, J. D. Radolf, and E. J. Hansen. 1997. Identification of tandem genes involved in lipooligosaccharide expression by Haemophilus ducreyi. Infect. Immun. 65(2): 651-660.

48. Süsskind, M., L. Brade, H. Brade, and O. Holst. 1998. Identification of a novel heptoglycan of α1-2 linked D-glycero-D-manno-heptopyrmnose. J. Biol. Chem. 273(12):7006-7017.

49. Taylor, D. E., D. A. Rasko, R. Sherburne, C. Ho, and L. D. Jewell. 1998. Lack of correlation between Lewis antigen expression by Helicobacter pylori and gastric epithelial cells in infected patients. Gastroenterology 115:1113-1122.

50. Thompson, J. D., D. G. Higgins, and T. J. Gibson. 1994. CLUSTAL W: improving the sensitivity of progressive multiple sequence alignment through sequence weighting, positions-specific gap penalties and weight matrix choice. Nucl. Acids Res. 22:4673-4680.

51. Toman, R. and L. _kultéty. 1995. Structural study on a lipopolysaccharide from Coxiella burnetii stain Nine Mile in avirulent phase II. Carbohydrate Res. 283:175-185.

52. Tomb J -F., O. White, A. R. Kerlavage, R. A. Clayton, G. G. Sutton, R. D. Fleischmann, K. A. Ketchum, H. P. Klenk, S. Gill, B. A. Dougherty, K. Nelson, J. Quackenbush, L. Zhou, E. F. Kirkness, S. Peterson, B. Loftus, D. Richarson, R. Dodson, H. G. Khalak, A. Glodek, K. McKenney, L. M. Fitzegerald, N. Lee, M. D. Adams, E. K. Hickey, D. E. Berg, J. D. Gocayne, T. R. Utterback, J. D. Peterson, J. M. Kelley, M. D. Cotton, J. M. Weidman, C. Fujii, C. Bowman, L. Watthey, E. Wallin, W. S. Hayes, M. Borodovsky, P. D. Karp, H. O. Smith, C. M. Fraser and J. C. Venter. 1997. The complete genome sequence of the gastric pathogen Helicobacter pylori. Nature 388:539-547.

53. Tsai, C. M., and C. E. Frasch. 1982. A sensitive silver stain for detecting lipopolysaccharide in polyacrylamide gels; Anal. Biochem. 119:115-119.

54. Wang, G., D. A. Rasko, R. Sherburne, and D. E. Taylor. 1999. Molecular genetic basis for the variable expression of Lewis Y antigen in Helicobacter pylori: analysis of the α(1,2) fucosyltransferase gene. Mol. Microbiol. 31(4):1265-1274.

55. Westphal, O. and Jann, K. 1965. Bacterial lipopolysaccharides. Extraction with phenol-water and further applications of the procedure. Meth. Carbohydr. Chem. 5: 83-91.

56. Yao, R., R. A. Alm, T. J. Trust, and P. Guerry. 1993. Construction of new Campylobacter cloning vectors and a new mutational cat cassette. Gene 130:127-130.

We claim:

1. An isolated DD-heptosyltransferase (DDhepT) polynucleotide of at least 30 nucleotides which hybridizes to SEQ ID NO. 1, 3, or 5 or the complement of SEQ ID NO. 1, 3, or 5 under stringent hybridization conditions.

2. An isolated DDhepT polynucleotide which comprises:
(a) a polynucleotide encoding a polypeptide having substantial sequence identity, preferably at least 50%, more preferably at least 70% sequence identity, with an amino acid sequence of SEQ. ID. NO. 2, 4, or 6;
(b) polynucleotides complementary to (a);
(c) polynucleotides differing from any of the polynucleotides of (a) or (b) in codon sequences due to the degeneracy of the genetic code;
(d) a polynucleotide comprising at least 10, 15, or 18, preferably at least 20 nucleotides and capable of hybridizing under stringent conditions to a polynucleotide of SEQ. ID. NO. 1, 3, or 5 or to a degenerate form thereof;
(e) a polynucleotide encoding an allelic or species variation of a polypeptide comprising an amino acid sequence of SEQ. ID. NO. 2, 4, or 6; or (f) a fragment, or allelic or species variation of (a), (b) or (c)

3. An isolated polynucleotide as claimed in claim 2 which comprises:
(a) a polynucleotide having substantial sequence identity, preferably at least 50%, more preferably at least 70% sequence identity with a sequence of SEQ. ID. NO. 1, 3, or 5;
(b) polynucleotides complementary to (a), preferably complementary to a full sequence of SEQ. ID. NO. 1, 3, or 5;
(c) polynucleotides differing from any of the polynucleotides of (a) to (b) in codon sequences due to the degeneracy of the genetic code; or
(d) a fragment, or allelic or species variation of (a), (b) or (c).

4. An isolated polynucleotide which encodes a polypeptide which binds an antibody of a DDHepT derived from Helicobacter pylori.

5. A vector comprising a polynucleotide of claim 1, 2, 3 or 4.

6. A host cell comprising a polynucleotide of any preceding claim.

7. An isolated DD-heptosyltransferase polypeptide comprising an amino acid sequence of SEQ. ID. NO. 2, 4, or 6.

8. An isolated polypeptide having at least 70% amino acid sequence identity to an amino acid sequence of SEQ. ID. NO. 2, 4, or 6.

9. A method for preparing a DDHepT polypeptide comprising an amino acid sequence of SEQ. ID. NO. 2, 4, or 6 comprising:
(a) transferring a vector as claimed in claim 5 into a host cell;
(b) selecting transformed host cells from untransformed host cells;
(c) culturing a selected transformed host cell under conditions which allow expression of the polypeptide; and
(d) isolating the polypeptide.

10. A recombinant polypeptide prepared in accordance with the method of claim 9.

11. An antibody having specificity against an epitope of a polypeptide as claimed in claim 7 or 8.

12. An antibody as claimed in claim 11 labeled with a detectable substance and used to detect the polypeptide in biological samples, tissues, and cells.

13. A probe comprising a sequence encoding a polypeptide as claimed in claim 7 or 8, or a part thereof.

14. A method of diagnosing and monitoring diseases by determining the presence of a polynucleotide or a polypeptide as claimed in any preceding claim.

15. A method for identifying a substance which associates with a polypeptide as claimed in claim 7 or 8 comprising (a) reacting the polypeptide with at least one substance which potentially can associate with the polypeptide, under conditions which permit the association between the substance and polypeptide, and (b) removing or detecting polypeptide associated with the substance, wherein detection of associated polypeptide and substance indicates the substance associates with the polypeptide.

16. A method as claimed in claim 15 wherein association of the polypeptide with the substance is detected by assaying for substance-polypeptide complexes, for free substance, for non-complexed polypeptide, for enzymatic activity of the polypeptide, or for activation of the polypeptide.

17. A method for identifying compounds which bind to or otherwise interact with and inhibit or activate an activity of a polypeptide or polynucleotide as claimed in any preceding claim comprising:
(a) contacting a polypeptide or polynucleotide as claimed in any preceding claim with a test compound under conditions to permit binding to or other interaction between the test compound and the polypeptide or polynucleotide to assess the binding to or other interaction with the test compound, wherein the binding or interaction is associated with a second component capable of providing a detectable signal in response to the binding or interaction of the polypeptide or polynucleotide with the test compound; and
(b) determining whether the test compound binds to or interacts with and activates or inhibits an activity of the polypeptide or polynucleotide by detecting the presence or absence of a signal generated from the binding or interaction of the test compound with the polypeptide or polynucleotide.

18. A method for evaluating a test compound for its ability to modulate the activity of a polypeptide as claimed in any preceding claim comprising (a) reacting an acceptor molecule and a donor molecule for the polypeptide in the presence of a test compound; (b) measuring transfer of a sugar of the donor molecule to the acceptor molecule, and (c) carrying out steps (a) and (b) in the absence of the test compound to determine if the compound interferes with or enhances transfer of the sugar of the donor molecule to the acceptor molecule by the polypeptide.

19. A method for formulating a pharmaceutical composition comprising
(a) conducting therapeutic profiling of test compounds identified in accordance with a method as claimed in claim 17 or 18, or further analogs thereof, for efficacy and toxicity in animals; and
(b) formulating a pharmaceutical composition including one or more test compounds identified in step (a) as having an acceptable therapeutic profile.

20. A method as claimed in claim 19 further comprising establishing a distribution system for distributing the pharmaceutical composition for sale, and optionally establishing a sales group for marketing the pharmaceutical composition.

21. A method of conducting a target discovery business comprising:
(a) providing a method as claimed in claim 17 or 18 for identifying test compounds that bind to or interact with and activate or inhibit or modulate an activity of the polypeptide or polynucleotide;
(b) optionally conducting therapeutic profiling of test compounds identified in (a) for efficacy and toxicity in animals; and
(c) licensing to a third party the rights for further drug development and/or sales for test compounds identified in step (a), or analogs thereof

22. A method for detecting a polynucleotide encoding a polypeptide comprising an amino acid sequence of SEQ. ID. NO. 2, 4, or 6 in a biological sample comprising the steps of:
(a) hybridizing a polynucleotide as claimed in any preceding claim to nucleic acids of the biological sample, thereby forming a hybridization complex; and
(b) detecting the hybridization complex wherein the presence of the hybridization complex correlates with the presence of a nucleic acid encoding the polypeptide in the biological sample.

23. A method as claimed in claim 22 wherein nucleic acids of the biological sample are amplified by the polymerase chain reaction prior to the hybridizing step.

24. A method for treating a disease comprising administering an effective amount of an antibody as claimed in claim 11 or a substance or compound identified in accordance with a method claimed in claim 15, 17 or 18.

25. A composition comprising one or more of a polynucleotide as claimed in any preceding claim or a polypeptide claimed in claim 7 or 8, and a pharmaceutically acceptable carrier, excipient or diluent.

26. A method for preparing an oligosaccharide comprising contacting a reaction mixture comprising an activated donor molecule, and an acceptor in the presence of a polypeptide as claimed in claim 7 or 8.

27. A mutant Helicobacter pylori having one or more inactivating mutations in a DDhepT gene which render the Helicobacter pylori avirulent.

28. A method for preparing an immunogenic composition comprising mixing a mutant Helicobacter pylori according to claim 27 with a pharmaceutically acceptable carrier.

29. An immunogenic composition for use in a human comprising a live avirulent derivative of Helicobacter pylori having one or more inactivating mutations in a DDhepT gene which render the Helicobacter pylori avirulent.

30. A mutant strain of H. pylori, said mutant strain having a deactivated DDhepT gene

31. A vaccine composition comprising an antigen derived from a mutant strain of H. pylori according to claim 27 or 30.

32. A vaccine composition according to claim 31, wherein the antigen is an at least partially purified lipopolysaccharide.

33. A vaccine composition according to claim 32, wherein the antigen is conjugated to a protein.

34. A live attenuated vaccine composition comprising a mutant strain of H. pylori according to claim 27 or 30.

35. A reaction mixture for an enzymatic synthesis of a Helicobacter lipopolysaccharide or a portion thereof the mixture comprising an isolated polypeptide as claimed in claim 7 or 8.

36. A reaction mixture according to claim 35, wherein the bacterial lipopolysaccharide is a mimic of a Helicobacter lipopolysaccharide.


SEQUENCE LISTING (14 sequences)

SEQ ID NO: 1 - DNA, 849 bp, Helicobacter pylori
  CDS (1)..(849)
atgcatgttgcttgtcttttggctttaggggataatctcatcacgcttagccttttaaaagaaatcgctttcaaacagcaacaaccccttaaaatcctaggtactcgtttgactttaaaaatcgccaagcttttagaatgcgaaaaacattttgaaatcattcctctttttgaaaatgtccctgctttttatgaccttaaaaaacaaggcgtttttttggcgatgaaggattttttatggttgttaaaagcgattaaaaagcatcaaatcaaacgtttgattttggaaaaacaggattttagaagcacttttttagccaaattcattcccataaccactccaaataaagaaattaaaaacgtttatcaaaaccgccaggagttgttttctcaaatttatgggcatgtttttgataaccccccatatcccatgaatttaaaaaaccccaaaaagattttgatcaaccctttcacaagatccatagaccgaagtatccctttagagcatttacaaatcgttttaaaacttttaaaacccttttgtgttacgcttttagattttgaagaacgatacgcttttttaaaagacagagtcgctcattatcgcgctaaaaccagtttagaagaagttaaaaacctgattttagaaagcgatttgtatataggaggggattcgtttttgatccatttggcttactatttaaagaaaaattattttatctttttttatagggataatgatgatttcatgccgcctaatagtaagaataaaaattttctaaaagcccacaaaagccattctatagaacaagatttagccaaaaaattccgccatttggggctattataa

SEQ ID NO: 2 - protein, 282 aa, Helicobacter pylori
MHVACLLALGDNLITLSLLKEIAFKQQQPLKILGTRLTLKIAKLLECEKHFEIIPLFENVPAFYDLKKQGVFLAMKDFLWLLKAIKKHQIKRLILEKQDFRSTFLAKFIPITTPNKEIKNVYQNRQELFSQIYGHVFDNPPYPMNLKNPKKILINPFTRSIDRSIPLEHLQIVLKLLKPFCVTLLDFEERYAFLKDRVAHYRAKTSLEEVKNLILESDLYIGGDSFLIHLAYYLKKNYFIFFYRDNDDFMPPNSKNKNFLKAHKSHSIEQDLAKKFRHLGLL

SEQ ID NO: 3 - DNA, 843 bp, Helicobacter pylori
  CDS (1)..(843)
atgcatgttgcttgtcttttggctttaggggataacctcatcacgcttagcctttgtgaagaaatcgctctcaaacagcaacaaccccttaaaatcctaggtactcgtttgactttaaaaatcgccaagcttttagaatgcgaaaaacattttgaaatcattcctgtttttaaaaatatccccgctttttatgaccttaaaaaacaaggcgttttttgggcgatgaaggattttttatggttattaaaagcgcttaagaagcacaaaatcaaacacttgattttagaaaaacaagattttagaagcgctcttttatccaaatttgtttccataaccactccaaataaagaaattaaaaatgcttatcaaaaccgccaggagttgttttctcaaatttatgggcatgtttttgataatagtcaatattccatgagtttaaaaaaccccaaaaagattttaatcaaccctttcacgagagaaaataatagaaatatttctttagaacatttgcaaatcgttttaaaactgttaaaacccttttgtgttacgcttttagattttgaagaacgatacgcttttttaaaagatgaagtcgctcattatcgcgctaaaaccagtttagaagaagctaaaaacctgattttagaaagcgatttgtatatagggggggattcgtttttgatccatttggcttactatttaaagaaaaattattttatctttttttatagggataatgacgatttcatgccgcctaagaatgaaaattttctaaaagcccataaaagccatttcatagagcaggatttagccacccagttccgccatttggggctattataa

SEQ ID NO: 4 - protein, 280 aa, Helicobacter pylori
MHVACLLALGDNLITLSLCEEIALKQQQPLKILGTRLTLKIAKLLECEKHFEIIPVFKNIPAFYDLKKQGVFWAMKDFLWLLKALKKHKIKHLILEKQDFRSALLSKFVSITTPNKEIKNAYQNRQELFSQIYGHVFDNSQYSMSLKNPKKILINPFTRENNRNISLEHLQIVLKLLKPFCVTLLDFEERYAFLKDEVAHYRAKTSLEEAKNLILESDLYIGGDSFLIHLAYYLKKNYFIFFYRDNDDFMPPKNENFLKAHKSHFIEQDLATQFRHLGLL

SEQ ID NO: 5 - DNA, 850 bp, Helicobacter pylori
  CDS (1)..(849)
atgcatgttgcttgtcttttggctttaggggataacctcatcacgcttagccttttaaaagaaatcgcttccaaacagcaacggccccttaaaatcctaggcactcgtttgactttaaaaatcgccaagcttttagaatgcgaaaaacattttgaaatcattcctatttttgaaaatatccctgctttttatgatcttaaaaaacaaggcgttttttgggcgatgaaggattttttatggttgttaaaagcaattaagaagcacaaaatcaaacatttgattttagaaaaacaagattttagaagttttcttttatccaaatttgtttccataaccactcccaataaagaaattaaaaacgtttatcaaaaccgccaggagttgttttctccaatttatgggcatgtttttgataaccccccatatcccatgaatttaaaaaaccccaaaaagattttgatcaaccctttcacaagatccatagagcgaagtatccctttagagcatttaaaaatcgttttaaaactcttaaaacccttttgtgttacgcttttagattttgaagaacgatacgcttttttacaaaatgaagccactcattatcgtgctaaaaccagtttagaagaagttaaaagcctgattttagaaagcgatttgtatatagggggggattcgtttttaatccatttggcttactatttaaagaaaaattattttatctttttttatagggataatgacgatttcatgccacctaatggtaagaaggaaaattttctaaaagcccacaaaagccattacatagaacaggatttagccaaaaaattccgccatttggggcttattataa

SEQ ID NO: 6 - protein, 283 aa, Helicobacter pylori
MHVACLLALGDNLITLSLLKEIASKQQRPLKILGTRLTLKIAKLLECEKHFEIIPIFENIPAFYDLKKQGVFWAMKDFLWLLKAIKKHKIKHLILEKQDFRSFLLSKFVSITTPNKEIKNVYQNRQELFSPIYGHVFDNPPYPMNLKNPKKILINPFTRSIERSIPLEHLKIVLKLLKPFCVTLLDFEERYAFLQNEATHYRAKTSLEEVKSLILESDLYIGGDSFLIHLAYYLKKNYFIFFYRDNDDFMPPNGKKENFLKAHKSHYIEQDLAKKFRHLGLII

SEQ ID NO: 7 - DNA, 18 bp, Artificial Sequence
  Primer, HP0479 - F1
gcctttatcaagctagag

SEQ ID NO: 8 - DNA, 19 bp, Artificial Sequence
  Primer, HP0479 - R1
cataaatgtcctaacaagc

SEQ ID NO: 9 - DNA, 18 bp, Artificial Sequence
  Primer, HP0479 - mutF1
caaaaccgccaggagttg

SEQ ID NO: 10 - DNA, 20 bp, Artificial Sequence
  Primer, HP0479-mutR1
ggttatgggaatgaatttgg

SEQ ID NO: 11 - DNA, 22 bp, Artificial Sequence
  Primer, HP0479-GF1
atgcatgttgcttgtcttttgg

SEQ ID NO: 12 - DNA, 20 bp, Artificial Sequence
  Primer, HP0479-GR1
ttataatagccccaaatggc

SEQ ID NO: 13 - DNA, 20 bp, Artificial Sequence
  Primer, HP0480-F1
gataacctcatcacgcttag

SEQ ID NO: 14 - DNA, 18 bp, Artificial Sequence
  Primer, HP0480-R1
ttcaatccattctaacgc